(19) United States
(12) Patent Application Publication
(10) Pub. No.: US 2022/0275444 A1
(43) Pub. Date: Sep. 1, 2022

(54) METHODS FOR GENERATING HIGH-DENSITY MAGNETIC DEVICES

(71) Applicant: 10X GENOMICS, INC., Pleasanton, CA (US)

(72) Inventor: Alireza SALMANZADEH, Pleasanton, CA (US)

(21) Appl. No.: 17/680,850

(22) Filed: Feb. 25, 2022

Related U.S. Application Data
(60) Provisional application No. 63/154,349, filed on Feb. 26, 2021.
(60) Provisional application No. 63/182,430, filed on Apr. 30, 2021.

(51) Int. Cl. C12Q 1/6874; C12Q 1/6841; C12Q 1/6837

(52) U.S. Cl. CPC C12Q 1/6874; C12Q 1/6841; C12Q 1/6837

(57) ABSTRACT

The present disclosure relates generally to devices and methods useful for generating high-density arrays of target features (e.g., beads) by a permanent magnet and a substrate comprising an array of trapping regions.

### CROSS-REFERENCE TO RELATED APPLICATIONS

This application claims the benefit of U.S. Provisional Patent Application No. 63154,349 filed Feb. 26, 2021, and U.S. Provisional Patent Application 63182,430 filed Apr. 30, 2021, both of which are incorporated herein by reference in their entirety.

### FIELD

The present disclosure relates generally to devices and methods useful for generating high-density arrays of target features (e.g., beads) by a permanent magnet and a substrate comprising an array of trapping regions.

### BACKGROUND

Magnetic separation is the process of using magnetic fields to separate components of a mixture that are magnetic in nature (e.g., ferromagnetic components, paramagnetic components, etc.). For example, the magnetic field can selectively apply a magnetic force on magnetic features in the mixture that can result in separation of the magnetic features from the non-magnetic features. Magnetic separation on a macro-scale (e.g., separation of macro-scale immunomagnetic assays) can be performed using small magnetic fields andor magnetic fields with small gradients that can be generated from widely available permanent magnets andor electromagnets.

It can be desirable to use magnetic separation on a micro-scale to generate a dense array of magnetic features. For example, micro-scale devices that can generate a micro-array of magnetic features can be integrated with existing microfluidic systems. Such integration can facilitate lab-on-a-chip technologies that can perform laboratory functions on an integrated circuitchip. However, in order to achieve micro-scale separation large magnetic fields andor magnetic fields with large gradients are needed (e.g., magnetic fields that are localized).

Accordingly, there remains a need in the art for methods and devices that allow for generation of an array of features on a surface by using strong magnetic fields andor magnetic fields with large gradients on a micro-scale. Additionally, there is a need that the methods and devices allow for integration with existing microfluidic systems.

### SUMMARY

This section provides a general summary of the disclosure, and is not comprehensive of its full scope or all of its features. In one aspect, provided herein are methods for preparing an array of target features including: (a) providing a substrate on or adjacent to a permanent magnet configured to generate a first magnetic field, the substrate comprising a first surface and a second surface, wherein the first surface comprises a plurality of magnetic micro-features arranged along the first surface, and wherein the second surface of the substrate is adjacent to the permanent magnet; (b) receiving, at the first surface of the substrate, a sample comprising a plurality of target features; (c) generating, by a magnetic micro-feature of the plurality of magnetic micro-features, a second magnetic field based on the interaction of the magnetic micro-feature with the first magnetic field; and (d) driving, by one or more of the first magnetic field and the second magnetic field, a target feature of the plurality of target features towards the magnetic micro-feature such that the target feature is associated with the magnetic micro-feature, thereby forming an array of the plurality of target features. In some embodiments, the magnetic micro-feature comprises an attachment moiety capable of binding to the target feature, and wherein association of the target feature with the magnetic micro-feature comprises binding of the target feature to the attachment moiety of the magnetic micro-feature. In some embodiments, the first surface comprises a plurality of micro-wells, and wherein a micro-well of the plurality of micro-wells comprises the magnetic micro-feature. In some embodiments, the magnetic micro-feature is one of a circular shape, a rectangular shape, a square shape, a triangular shape and a star shape. In some embodiments, the plurality of magnetic micro-features is arranged along the first surface in a rectangular array. In some embodiments, a first gradient associated with the first magnetic field is smaller than a second gradient associated with the second magnetic field at the magnetic micro-feature. In some embodiments, the arrangement of micro features may be a repeating pattern with substantially identical inter-feature distances. In some embodiments, the micro features may be patterned with randomindiscriminate inter-feature distances.

In some embodiments, the plurality of the magnetic micro-features comprise a chemical coating on the surface of the magnetic micro-features. In some embodiments, the plurality of the micro-wells comprise a chemical coating on the surface of the micro-wells.

In some embodiments, the methods of the disclosure further include coupling the substrate with a microfluidic system, wherein the sample comprising the plurality of target features is received from the microfluidic system. In some embodiments, the magnetic micro-feature is a ferromagnetic micro-feature. In some embodiments, the magnetic micro-feature comprises one or more of Ni and NiFe. In some embodiments, the target feature is a magnetic bead or a bead coupled to a magnetic moiety. In some embodiments, the first surface is substantially parallel to the second surface. In some embodiments, the methods of the disclosure further include fabricating the substrate comprising the plurality of micro-features, the fabricating comprising one of template electrodeposition and electroplating. In some embodiments, the target feature comprises a capture probe, wherein the capture probe comprises a spatial barcode and a capture domain, wherein the capture domain is capable of binding to an analyte, and wherein the spatial barcode is different for each target feature of the plurality of target features.

In some embodiments, the target feature has a location on the substrate, and wherein the method further comprises associating the target feature with its location on the substrate. In some embodiments, the associating step includes determining the sequence of the spatial barcode of the target feature and associating the determined spatial barcode sequence with the location of the target feature on the substrate. In some embodiments, the determining step comprises sequencing. In some embodiments, the sequencing is in situ sequencing. In some embodiments, in situ sequencing is performed via sequencing-by-synthesis (SBS), sequential fluorescence hybridization, sequencing by ligation, nucleic acid hybridization, or high-throughput digital sequencing techniques.

In one aspect, provided herein are devices including (a) a substrate comprising a first surface and a second surface, wherein the first surface comprises a plurality of magnetic micro-features arranged along the first surface, and wherein the first surface is configured to receive a sample comprising a plurality of target features; and (b) a permanent magnet configured to couple to the second surface of the substrate and to generate a first magnetic field, wherein a magnetic micro-feature of the plurality of magnetic micro-features is configured to generate a second magnetic field based on the interaction of the magnetic micro-feature with the first magnetic field, and (c) wherein one or more of the first magnetic field and the second magnetic field are configured to drive a target feature of the plurality of target features towards the magnetic micro-feature such that the target feature is associated with the magnetic micro-feature, thereby forming an array of the plurality of target features.In some embodiments, the magnetic micro-feature comprises an attachment moiety capable of binding to the target feature, and wherein association of the target feature with the magnetic micro-feature comprises binding of the target feature to the attachment moiety of the magnetic micro-feature. In some embodiments, wherein the first surface comprises a plurality of micro-wells, and wherein a micro-well of the plurality of micro-wells comprises the magnetic micro-feature. In some embodiments, the magnetic micro-feature is one of a circular shape, a rectangular shape, a square shape, a triangular shape and a star shape. In some embodiments, the plurality of magnetic micro-features are arranged along the first surface in a rectangular array. In some embodiments, a first gradient associated with the first magnetic field is smaller than a second gradient associated with the second magnetic field at the magnetic micro-feature. In some embodiments, the substrate is configured to couple with a microfluidic system, wherein the sample comprising the plurality of target features is received from the microfluidic system. In some embodiments, the arrangement of micro features is a repeating pattern with substantially identical inter-feature distances. In some embodiments, micro features are patterned with randomindiscriminate inter-feature distances.

In some embodiments, the magnetic micro-feature is a ferromagnetic micro-feature. In some embodiments, the magnetic micro-feature comprises one or more of Ni and NiFe. In some embodiments, the target feature is a magnetic bead or a bead coupled to a magnetic moiety. In some embodiments, the first surface is substantially parallel to the second surface. In some embodiments, the target feature further comprises a capture probe, wherein the capture probe comprises a spatial barcode and a capture domain, wherein the capture domain is capable of binding to an analyte, and the spatial barcode is different for each target feature of the plurality of target features. In some embodiments, the capture probe comprises an adapter region.

In one aspect, provided herein are devices including (a) a substrate comprising a first surface and a second surface, wherein the first surface comprises a plurality micro-wells arranged along the first surface, and wherein the first surface is configured to receive a sample comprising a plurality of target features; and (b) a permanent magnet configured to couple to the second surface of the substrate and to generate a first magnetic field, (c) wherein the first magnetic field is configured to drive a target feature of the plurality of target features towards a micro-well of the plurality of micro-wells such that the target feature is associated with the micro-well, thereby forming an array of the plurality of target features.

In some embodiments, the micro-well comprises an attachment moiety capable of binding to the target feature, and wherein association of the target feature with the micro-well comprises binding of the target feature to the attachment moiety of the micro-well. In some embodiments, a micro-well of the plurality of micro-wells includes a magnetic micro-feature attached to a surface of the micro-well. In some embodiments, the plurality of micro-wells are arranged along the first surface in a rectangular array. In some embodiments, the substrate is configured to couple with a microfluidic system, wherein the sample comprising the plurality of target features is received from the microfluidic system.

In some embodiments, the magnetic micro-feature is a ferromagnetic micro-feature. In some embodiments, the magnetic micro-feature comprises one or more of Ni and NiFe. In some embodiments, the target feature is a magnetic bead or a bead coupled to a magnetic moiety. In some embodiments, the first surface is substantially parallel to the second surface. In some embodiments, the target feature further comprises a capture probe, wherein the capture probe comprises a spatial barcode and a capture domain, wherein the capture domain is capable of binding to an analyte, and wherein the spatial barcode is different for each target feature of the plurality of target features. In some embodiments, the capture probe comprises an adapter region.

In one aspect, provided herein are devices including spatial arrays prepared by any combination of methods described above.

In one aspect, provided herein are methods for spatial analysis of a biological analyte in a biological sample including (a) providing an array prepared by any combination of methods described above; (b) contacting the biological sample to the array under conditions wherein the biological analyte binds the capture probe on the target feature; and (c) determining a location of the analyte on the surface of the substrate based on the binding of the analyte to the capture probe, and using the location of the analyte on the surface of the substrate to identify the location of the analyte in the biological sample.

In one aspect, provided herein are methods for spatial analysis of a biological analyte in a biological sample including (a) providing an array prepared by any combination of methods described above; (b) contacting the biological sample to the array under conditions wherein the biological analyte binds the capture probe on the target feature; and (c) determining (i) all or a part of the sequence of the biological analyte specifically bound to the capture domain, or a complement thereof; and (ii) the sequence of the spatial barcode, or a complement thereof, and using the determined sequence of (i) and (ii) to identify the location of the analyte in the biological sample.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIG. 1 schematically illustrates an exemplary method for generating an array of target features on a first surface of a substrate that includes magnetic micro-features arranged in an array.

FIG. 2 schematically illustrates the exemplary method of FIG. 1 that further includes a chemical coating on the surface of magnetic micro-features that can immobilize the target feature over the magnetic micro-features.

FIG. 3 schematically illustrates an exemplary method for generating an array of target features on a first surface of a substrate that includes multiple trapping regions including micro-wells arranged in an array.

FIG. 4 schematically illustrates the exemplary method of FIG. 3 that further includes magnetic micro-features in the micro-wells.

FIG. 5 schematically illustrates the exemplary method of FIG. 3 that further includes chemical coating on a surface of the micro-wells.

### DETAILED DESCRIPTION OF THE DISCLOSURE

The present disclosure generally relates to, inter alfa, methods for generating devices containing high-density arrangements of target features (e.g., arrays with high-density arrangements of magnetic features). As described in greater detail below, microfluidic-based magnetic separation has the capability and advantage of integrability with other microfluidic and lab-on-a-chip technologies (system integration). The arrays disclosed herein are generated not only by a permanent magnet, but also by patterning micro-features on a substrate, which can create a localized magnetic field gradient, and consequently, stronger magnetic force.

In contrast, the conventional macro-scale immuno-magnetic assays, generated by permanent magnets, have low gradient of magnetic field (low magnetic force on magnetic features) and low trapping efficiency gradient. The conventional macro-scale assays cannot be integrated with microfluidic and lab-on-chip technologies

Magnetic force is proportional with the gradient of magnetic field. Which is provided by the ferromagnetic micro-features. Very high-density array can be made through this approach since ferromagnetic micro-features can be fabricated in very high-density by conventional MEMS fabrication technologies, e.g., devices that integrate mechanical systems with electronic circuits (MEMS: Microelectromechanical Systems).

Some embodiments of the present disclosure relate to a novel approach where a permanent magnet is used to attract the magnetic features (e.g. magnetically-labeled biological particles, such as cells, cell beads, or nuclei, through magnetizing, magnetic beads, etc.) toward the surface containing micro-features. The permanent magnet also magnetizes the ferromagnetic micro-features on the surface of the substrate. The strong gradient and magnetic force generated by the ferromagnetic micro-features captures the magnetically-labeled biological particles.

As also described in greater detail below, there are several parameters that one can suitably use to optimize the magnetic force in designing ferromagnetic micro-features, such as for example size, dimensions, material, distance between them, and their arrangement. For example, ferromagnetic micro-features can be designed with different geometries such as circular, rectangular, square, triangular, etc. In addition or alternatively, ferromagnetic micro-features can be designed in wide range of dimensions depending on the desired density of the array. Furthermore, magnetic features (e.g., magnetic beads or magnetically-labeled biological features such as cells or cell beads) that are magnetically trapped on an array can be immobilized by chemical coating the surface of trapping spots on the array. For instance, biological particles (e.g, cells or nuclei) can be immobilized by biotin-streptavidin affinity binding. Moreover, the ferromagnetic micro-features can be fabricated from any ferromagnetic material which could be micro-patterned on a substrate (such as glass or Silicon) through conventional technologies. Common materials include, but are not limited to Ni and NiFe.

Accordingly, some embodiments of the present disclosure relate to methods for generating a high-density array of target features such as, magnetic features (e.g., magnetic beads or magnetically-labeled biological particles, such as cells, cell beads, or nuclei) on a surface of a substrate. The substrate can be integrated with a microfluidic system that can transport a sample including the target features onto a first surface of the substrate. In particular, the first surface of the substrate can include an array of trapping regions (e.g., magnetic micro-features, micro-wells, chemical coatings or a combination thereof). A second surface of the substrate (e.g., parallel to the first surface) can be positioned in proximity (e.g., adjacent) to a permanent magnet. The target features can be driven to the trapping regions (e.g., by a first magnetic field of the permanent magnet andor magnetic field generated by interaction of trapping regions with the first magnetic field) and captured. This can result in the formation of an array of target features. A high-density array of trapping regions can result in a high-density array of target features.

In some embodiments, the trapping regions on the surface of the substrate can include magnetic micro-features (e.g., ferromagnetic micro-features) that can interact with the first magnetic field of the permanent magnet and generate a second magnetic field. A magnetic force resulting from the interaction between the first andor the second magnetic field and the target features can drive the target features to the magnetic micro-features. In some embodiments, the trapping regions can include micro-wells (e.g., etched onto the surface of the substrate), and the target features can be driven into the micro-wells (e.g., by the first magnetic field, gravity, etc.). In some embodiments, the micro-wells can further include magnetic micro-features. In some embodiments the trapping regions can include a chemical coating that can immobilize the target features over the trapping region (e.g., after the target features are driven to the trapping region by the first andor second magnetic fields).

The applicant has developed a device that includes a substrate including a high-density array of trapping regions arranged on a surface of the substrate. The high-density array of trapping regions can be fabricated on the surface of the substrate by conventional fabrication techniques (e.g., MEMS fabrication technologies, electroplating, template electrodeposition, etc.), and can generate a localized high-gradient magnetic field that can capturetrap the target features. This can result in an array of high-density target features. The trapping of the target features can be varied (e.g., optimized) by varying the properties (e.g., size, shape, material, periodicity, geometry, etc.) of the trapping regions.

In the following detailed description, reference is made to the accompanying drawings, which form a part hereof. In the drawings, similar symbols generally identify similar components, unless context dictates otherwise. The illustrative alternatives described in the detailed description, drawings, and claims are not meant to be limiting. Other alternatives may be used and other changes may be made without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of this application.

Unless otherwise defined, all terms of art, notations, and other scientific terms or terminology used herein are intended to have the meanings commonly understood by those of skill in the art to which this application pertains. In some cases, terms with commonly understood meanings are defined herein for clarity andor for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity andor for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, including mixtures thereof “A andor B” is used herein to include all of the following alternatives: “A”, “B”, “A or B”, and “A and B”.

An “adapter,” an “adaptor,” and a “tag” are terms that are used interchangeably in this disclosure, and refer to moieties that can be coupled to a polynucleotide sequence (in a process referred to as “tagging”) using any one of many different techniques including (but not limited to) ligation, hybridization, and tagmentation. Adapters can also be nucleic acid sequences that add a function, e.g., spacer sequences, primer sequences, primer binding sites, barcode sequences, and unique molecular identifier sequences.

The term “barcode” is used herein to refer to a label, or identifier, that conveys or is capable of conveying information (e.g., information about an analyte in a sample, a bead, andor a capture probe). A barcode can be part of an analyte or capture probe, or independent of an analyte or capture probe. A barcode can be attached to an analyte or capture probe in a reversible or irreversible manner. A particular barcode can be unique relative to other barcodes. Barcodes can have a variety of different formats. For example, barcodes can include polynucleotide barcodes, random nucleic acid andor amino acid sequences, and synthetic nucleic acid andor amino acid sequences. A barcode can be attached to an analyte or to another moiety or structure in a reversible or irreversible manner. A barcode can be added to, for example, a fragment of a deoxyribo-nucleic acid (DNA) or ribonucleic acid (RNA) sample before or during sequencing of the sample. Barcodes can allow for or facilitates identification andor quantification of individual sequencing-reads. Barcodes can spatially-resolve molecular components found in biological samples, for example, at single-cell (or single nucleus) resolution (e.g., a barcode can be or can include a “spatial barcode”). In some embodiments, a barcode includes two or more sub-barcodes that together function as a single barcode. For example, a polynucleotide barcode can include two or more polynucleotide sequences (e.g., sub-barcodes) that are separated by one or more non-barcode sequences.

The term “percent identity,” as used herein in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See e.g., the NCBI web site at ncbi.nlm.nih.govBLAST. Such sequences are then said to be “substantially identical.” This definition also refers to, or may be applied to, the complement of a sequence. This definition also includes sequences that have deletions andor additions, as well as those that have substitutions. Sequence identity can be calculated using published techniques and widely available computer programs, such as the GCS program package (Devereux et al, Nucleic Acids Res. 12:387, 1984), BLASTP, BLASTN, FASTA (Atschul et al., J Mol Biol 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

It is understood that aspects and embodiments of the disclosure described herein include “comprising”, “consisting”, and “consisting essentially of” aspects and embodiments.

As used herein, “comprising” is synonymous with “including”, “containing”, or “characterized by”, and is inclusive or open-ended and does not exclude additional, unrecited elements or method steps. Any recitation herein of the term “comprising”, particularly in a description of components of a composition or in a description of steps of a method, is understood to encompass those compositions and methods consisting essentially of and consisting of the recited components or steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method.

Where a range of values is provided, it is understood by one having ordinary skill in the art that all ranges disclosed herein encompass any and all possible sub-ranges and combinations of sub-ranges thereof Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to”, “at least”, “greater than”, “less than”, and the like include the number recited and refer to ranges which can be subsequently broken down into sub-ranges as dis-cussed above. As will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value.

The term “biological particle,” as used herein, generally refers to a discrete biological system derived from a biological sample. The biological particle may be a macromolecule, small molecule, virus, cell, cell derivative, cell nucleus, cell organelle, cell constituent and the like. Examples of a cell organelle include, without limitation, a nucleus, endoplasmic reticulum, a ribosome, a Golgi apparatus, an endoplasmic reticulum, a chloroplast, an endocytic vesicle, an exocytic vesicle, a vacuole, and a lysosome. The biological particle may contain multiple individual components, such as macromolecules, small molecules, viruses, cells, cell derivatives, cell nuclei, cell organelles and cell constituents, including combinations of different of these and other components. The biological particle may be or may include DNA, RNA, organelles, proteins, or any combination thereof. These components may be extracellular. In some examples, the biological particle may be referred to as a clump or aggregate of combinations of components. In some instances, the biological particle may include one or more constituents of a cell but may not include other constituents of the cell. An example of such constituents include nucleus or an organelle. A cell may be a live or viable cell. The live cell may be capable of being cultured, for example, being cultured when enclosed in a gel or polymer matrix or cultured when comprising a gel or polymer matrix. A biological particle may include a single cell andor a single nuclei from a cell. The biological particle may be or may include a matrix (e.g., a gel or polymer matrix) comprising a cell or one or more constituents from a cell (e.g., cell bead), such as DNA, RNA, organelles, proteins, or any combination thereof, from the cell. The biological particle may be obtained from a tissue of a subject. The biological particle may be a hardened cell. Such hardened cell may or may not include a cell wall or cell membrane. The biological particle may include one or more constituents of a cell, but may not include other constituents of the cell. An example of such constituents is a nucleus or an organelle.

Headings, e.g., (a), (b), (i) etc., are presented merely for ease of reading the specification and claims. The use of headings in the specification or claims does not require the steps or elements be performed in alphabetical or numerical order or the order in which they are presented.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### Devices of the Disclosure

As outlined above, some aspects of the disclosure provide new devices designed for magnetically directing and capturingtrapping magnetic features on a surface of a substrate. In particular, some embodiments of the disclosure relate to devices that include magnetic micro-features (e.g., ferromagnetic micro-features) andor micro-wells (e.g., etched into) the surface of the substrate.

In some embodiments, the devices of the disclosure can include a substrate and a permanent magnet. The substrate can include a first surface and a second surface (e.g., parallel to the first surface). Multiple trapping regions can be arranged along the first surface (e.g. forming an array of trapping regions). In some embodiments, the trapping regions can include magnetic micro-features (e.g., ferromagnetic micro-features). The magnetic micro-features can be fabricated on the first surface (e.g., by electroplating, template electrodeposition, etc.). The permanent magnet can be configured to be placed in proximity to (or adjacent to) the second surface of the substrate. Examples of suitable substrates that can be used in the devices and methods described herein include, but are not limited to, slides (e.g., slides formed from various glasses, slides formed from various polymers), hydrogels, layers andor films, membranes (e.g., porous membranes), cuvettes, wafers, plates, or combinations thereof. Various embodiments of substrate and their properties are described below.

The first surface be configured to receive a sample including a plurality of target features. In some embodiments, the substrate can be coupled (e.g., releasably attached) to a microfluidic system that can transport the sample. Examples of suitable microfluidic system that can be used in the devices and methods described herein include, but are not limited to, channels, reservoirs, valves, seals, etc.

One or more magnetic micro-features can be configured to generate a second magnetic field based on their interaction with the first magnetic field of the permanent magnet. The magnetic micro-features can include ferromagnetic material that can get magnetized in the presence of an external magnetic field (e.g., first magnetic field of the permanent magnet). The magnetization can involve alignment of magnetic dipoles in the magnetic micro-features that can result in the generation of the second magnetic field. The second magnetic field can be localized around the micro-features. In other words, the strength of the second magnetic field can have large spatial variation (e.g., relative to the spatial variation of the strength of first magnetic field) near the magnetic micro-features (e.g., on the surface of the micro-feature). The second magnetic field can have a higher gradient than the first magnetic field near the magnetic micro-features, and may apply a stronger magnetic force on a magnetic feature (e.g., a magnetic target feature) relative to the magnetic force applied by the first magnetic field.

In some embodiments, the magnetic micro-features can be ferromagnetic or can include one or more ferromagnetic materials (e.g., Ni, NiFe, etc.). In some embodiments, the micro-features can be arranged in various patterns (e.g., a rectangular array, a square array, a random pattern, etc.). Various embodiments of arrays and their properties are described below. The micro-features can have various shapes (e.g., a circular shape, a rectangular shape, a square shape, a triangular shape, a star shape, etc.). In some embodiments, the micro-features can have random shapes (e.g., random shapes resulting from the fabrication of the micro-features). In some embodiments, the spacing between the micro-features (center-to-center distance) can be between 3 microns and 10 microns, between 4 microns and 9 microns, between 5 microns and 8 microns, between 6 microns and 7 microns. In some embodiments, the spacing between micro-features can be less than 50 microns (center-to-center distance), less than 60 microns, less than 70 microns, less than 80 microns, less than 90 microns, less than 100 microns or any values in between.

In some embodiments, the arrangement of micro-features may be a repeating pattern with substantially identical inter-feature distances. Also conversely, micro-features may be patterned with randomindiscriminate inter-feature distances.

In some embodiments, the target features can include magnetic beads. Magnetic beads can include beads that wholly or partially include a magnetic material (e.g., ferromagnetic material, paramagnetic material, diamagnetic material, etc.). Various embodiments of beads and properties thereof are described below. In some embodiments, a magnetic bead can include a bead coupled to a magnetic moiety. A description in greater detail of beads suitable for the devices and methods of the disclosure is further provided below.

The magnetic beads can interact with an external magnetic field (e.g., first magnetic field andor second magnetic field) and as a result experience a magnetic force. The strength of the magnetic force can depend on the strength of the external magnetic field andor the gradient of external magnetic field. For example, the strength of the magnetic force can depend on the magnetization of the magnetic beads which can depend on the strength of the external magnetic field. The strength of the magnetic field can also depend on the gradient of the external magnetic field.

A target feature (of a plurality of target feature) can experience a magnetic force due to the first magnetic field from the permanent magnet andor the second magnetic field from one or more magnetic micro-features. The magnetic force can drive the target feature towards a magnetic micro-feature such that the target feature is associated with the magnetic micro-feature (e.g., releasably attached to the magnetic micro-feature). For example, the first magnetic field from the permanent magnet can attract the target feature towards the first surface of the substrate (e.g., when the second magnetic field is much weaker than the first magnetic field). When the target feature is sufficiently close to the magnetic micro-feature (e.g., at a distance from the magnetic micro-feature where the gradient of the second magnetic field generated by the magnetic micro-feature is much larger than the gradient of the first magnetic field), it can experience a magnetic force resulting from its interaction with the second magnetic field. This magnetic force can be strong (e.g., due to high gradient of the second magnetic field) and can capture the target feature at the magnetic micro-feature.

Other target features (of the plurality of target features) can be magnetically driven to and captured by the various magnetic micro-features. This can result in a spatial array of the plurality of target features. In some embodiments, multiple target features can be attached to one magnetic micro-feature. Additionally or alternately, a target feature can be attached to multiple magnetic micro-features. The density of the array of target features can be proportional the density of magnetic micro-features. A substrate with a high-density array of magnetic micro-features can result in a high-density array of target features. As described above, the target features can include beads that are magnetic. Various embodiments and properties of an array target features (or “bead arrays”) on a substrate are described below.

In some embodiments, the magnetic micro-features can include a chemical coating that can immobilize the target features at the magnetic micro-features. In some embodiments, the chemical coating of the magnetic micro-feature can include an attachment moiety capable of binding to and immobilizing the target feature. In some embodiments, coatings can include a polymer that can be activated (e.g., photochemically, chemically, etc.) to capture the target features. Various embodiments of coatings and their properties are described below. Immobilizing the target features can generate a structurally stable array of target features. For example, the array of target features may remain intact on the first surface of the substrate when the permanent magnet is removed (or the first magnetic field attenuated).

In some embodiments, trapping regions can include micro-wells (e.g., etched) on the first surface of the substrate. For example, the first surface of the substrate can include an array of micro-wells. One or more target features can be driven by the first magnetic field into a micro-well and captured therein. In some embodiments, the micro-wells can include magnetic micro-features that can generate a second magnetic field as described above. In some embodiments, the micro-wells can include a chemical coating that can immobilize the captured target feature. In some embodiments, the chemical coating can be overlaid on the magnetic micro-feature in the micro-well. In some embodiments, the walls of the micro-wells can include the chemical coating. In some embodiments, chemical coatings can be overlaid on the base of the micro-wells (e.g., portion of the micro-well parallel to the first surface of the substrate).

In some embodiments, the target features can include a capture probe. In some embodiments, the capture probe can include one or more of a spatial barcode, a unique molecular identifier (UMI), and a capture domain. The capture probe can be capable of capturing andor labelling an analyte of a biological sample. In some embodiments, the capture domain can include an anchor (e.g., sequence of nucleotides) that can allow the capture domain to hybridize with an intended analyte. In some embodiments, the capture probe can include an adapter (e.g, to allow for downstream analysis of analytes captured by the capture probe). In some embodiments, each target feature can have a unique spatial barcode. The spatial barcode can be used to identify the target feature (e.g., identify the location of a target feature in the array of target features on the first surface of the substrate). Various embodiments and properties of capture probe, anchor, adapter, spatial barcode, capture domain and UMI are described below.

The devices described herein include a plurality of features, which are each designed andor configured as a support or repository for various molecular entities used in sample analysis. In some embodiments, some or all of the features in an array are functionalized for analyte capture. In some embodiments, functionalized features include one or more capture probe(s). Examples of features include, but are not limited to, beads and microspheres. In some embodiments, features are directly or indirectly attached or fixed to a substrate. In some embodiments, the features are not directly or indirectly attached or fixed to a substrate, but instead, for example, are disposed within an enclosed or partially enclosed three dimensional space (e.g., wells or divots).

As another example, in some embodiments, features are formed by metallic micro-or nanoparticles. Suitable methods for depositing such particles to form arrays are described, for example, in Lee et al., Beilstein J. Nanotechnol. 8: 1049-1055 (2017).

As another example, in some embodiments, features correspond to regions of a substrate in which one or more optical labels have been incorporated, andor which have been altered by a process such as permanent photobleaching. Suitable substrates to implement features in this manner include a wide variety of polymers, for example. Methods for forming such features are described, for example, in Moshrefzadeh et al., Appl. Phys. Lett. 62: 16 (1993).

As yet another example, in some embodiments, features can correspond to colloidal particles assembled (e.g., via self-assembly) to form an array. Suitable colloidal particles are described for example in Sharma, Resonance 23(3): 263-275 (2018).

As a further example, in some embodiments, features can be formed via spot-array photopolymerization of a monomer solution on a substrate. In particular, two-photon and three-photon polymerization can be used to fabricate features of relatively small (e.g., sub-micron) dimensions. Suitable methods for preparing features on a substrate in this manner are described for example in Nguyen et al., Materials Today 20(6): 314-322 (2017).

In some embodiments, features are directly or indirectly attached or fixed to a substrate that is liquid permeable. In some embodiments, features are directly or indirectly attached or fixed to a substrate that is biocompatible. In some embodiments, features are directly or indirectly attached or fixed to a substrate that is a hydrogel.

As a further example, in some embodiments, features are formed by magnetic features that are assembled on a substrate. Examples of such features and methods for assembling arrays are described in Ye et al., Scientific Reports 6: 23145 (2016). Accordingly, in some embodiments, the devices of the disclosure include a plurality of magnetic features. In some embodiments, the at least a portion of the plurality of magnetic features comprise a plurality of magnetic beads having at least one oligonucleotide attached to a surface thereof.

In some embodiments, the at least a portion of the plurality of magnetic features comprise a plurality of non-magnetic beads having at least one magnetically-labeled oligonucleotide attached to a surface thereof.

In some embodiments, the magnetic features can be magnetic biological features, such as magnetically-labeled biological particles (e.g., cells, cell beads, or nuclei), or magnetically-labeled macromolecular constituents of a cell.

Substrate

For the methods of generating arrays described herein, a substrate is designed andor configured to function as a support for direct or indirect attachment of capture probes to features of the array. In addition, in some embodiments, a substrate (e.g., the same substrate or a different substrate) can be used to provide support to a biological sample, particularly, for example, a thin tissue section. Accordingly, a “substrate” is a support that is insoluble in aqueous liquid and which allows for positioning of biological samples, analytes, features, andor capture probes on the substrate.

Further, a “substrate” as used herein, and when not preceded by the modifier “chemical”, refers to a member with at least one surface that generally functions to provide physical support for biological samples, analytes, andor any of the other chemical andor physical moieties, agents, and structures described herein. Substrates can be formed from a variety of solid materials, gel-based materials, colloidal materials, semi-solid materials (e.g., materials that are at least partially cross-linked), materials that are fully or partially cured, and materials that undergo a phase change or transition to provide physical support. Examples of substrates that can be used in the methods and devices described herein include, but are not limited to, slides (e.g., slides formed from various glasses, slides formed from various polymers), hydrogels, layers andor films, membranes (e.g., porous membranes), flow cells, cuvettes, wafers, plates, or combinations thereof. In some embodiments, substrates can optionally include functional elements such as recesses, protruding structures, microfluidic elements (e.g., channels, reservoirs, electrodes, valves, seals), and various markings, as will be discussed in further detail below.

A substrate can generally have any suitable form or format. For example, a substrate can be flat, curved, e.g., convexly or concavely curved towards the area where the interaction between a biological sample, e.g., tissue sample, and a substrate takes place. In some embodiments, a substrate is flat, e.g., planar, chip, or slide. A substrate can contain one or more patterned surfaces within the substrate (e.g., channels, wells, projections, ridges, divots, etc.).

A substrate can be of any desired shape. For example, a substrate can be generally a thin, flat shape (e.g., a square or a rectangle). In some embodiments, a substrate structure has rounded corners (e.g., for increased safety or robustness). In some embodiments, a substrate structure has one or more cut-off corners (e.g., for use with a slide clamp or cross-table). In some embodiments, where a substrate structure is flat, the substrate structure can be any appropriate type of support having a flat surface (e.g., a chip or a slide such as a microscope slide).

Substrates can optionally include various structures such as, but not limited to, projections, ridges, and channels. A substrate can be micropatterned to limit lateral diffusion (e.g., to prevent overlap of spatial barcodes). A substrate modified with such structures can be modified to allow association of analytes, features (e.g., beads), or probes at individual sites. For example, the sites where a substrate is modified with various structures can be contiguous or non-contiguous with other sites.

In some embodiments, the surface of a substrate can be modified so that discrete sites are formed that can only have or accommodate a single feature. In some embodiments, the surface of a substrate can be modified so that features adhere to random sites.

In some embodiments, the surface of a substrate is modified to contain one or more wells, using techniques such as (but not limited to) stamping, microetching, or molding techniques. In some embodiments in which a substrate includes one or more wells, the substrate can be a concavity slide or cavity slide. For example, wells can be formed by one or more shallow depressions on the surface of the substrate. In some embodiments, where a substrate includes one or more wells, the wells can be formed by attaching a cassette (e.g., a cassette containing one or more chambers) to a surface of the substrate structure.

In some embodiments, the structures of a substrate (e.g., wells or features) can each bear a different capture probe. Different capture probes attached to each structure can be identified according to the locations of the structures in or on the surface of the substrate. Exemplary substrates include arrays in which separate structures are located on the substrate including, for example, those having wells that accommodate features.

In some embodiments where the substrate is modified to contain one or more structures, including but not limited to, wells, projections, ridges, features, or markings, the structures can include physically altered sites. For example, a substrate modified with various structures can include physical properties, including, but not limited to, physical configurations, magnetic or compressive forces, chemically functionalized sites, chemically altered sites, andor electrostatically altered sites. In some embodiments where the substrate is modified to contain various structures, including but not limited to wells, projections, ridges, features, or markings, the structures are applied in a pattern. Alternatively, the structures can be randomly distributed.

A wide variety of different substrates can be used for the foregoing purposes. In general, a substrate can be any suitable support material. Exemplary substrates include, but are not limited to, glass, modified andor functionalized glass, hydrogels, films, membranes, plastics (including e.g., acrylics, polystyrene, copolymers of styrene and other materials, polypropylene, polyethylene, polybutylene, polyurethanes, Teflon™, cyclic olefins, polyimides etc.), nylon, ceramics, resins, Zeonor, silica or silica-based materials including silicon and modified silicon, carbon, metals, inorganic glasses, optical fiber bundles, and polymers, such as polystyrene, cyclic olefin copolymers (COCs), cyclic olefin polymers (COPs), polypropylene, polyethylene polycarbonate, or combinations thereof

In another example, a substrate can be a flow cell. Flow cells can be formed of any of the foregoing materials, and can include channels that permit reagents, solvents, features, and analytes to pass through the flow cell. In some embodiments, a hydrogel embedded biological sample is assembled in a flow cell (e.g., the flow cell is utilized to introduce the hydrogel to the biological sample). In some embodiments, a hydrogel embedded biological sample is not assembled in a flow cell. In some embodiments, the hydrogel embedded biological sample can then be prepared andor isometrically expanded as described herein.

In some embodiments, the substrate is a conductive substrate. Conductive substrates (e.g., electrophoretic compatible arrays) generated as described herein can be used in the spatial detection of analytes. For example, an electrophoretic field can be applied to facilitate migration of analytes towards the barcoded oligonucleotides (e.g., capture probes) on the array (e.g., capture probes immobilized on paper, capture probes immobilized in a hydrogel film, or capture probes immobilized on a glass slide having a conductive coating). In some embodiments, a conductive substrate can include glass (e.g., a glass slide) that has been coated with a substance or otherwise modified to confer conductive properties to the glass.

Arrays

As described herein, features (as described further below) are collectively positioned on a substrate. An “array” is a specific arrangement of a plurality of features that is either irregular or forms a regular pattern. Individual features in the array differ from one another based on their relative spatial locations. In general, at least two of the plurality of features in the array include a distinct capture probe (e.g., any of the examples of capture probes described herein).

Arrays can be used to measure large numbers of analytes simultaneously. In some embodiments, oligonucleotides are used, at least in part, to create an array. For example, one or more copies of a single species of oligonucleotide (e.g., capture probe) can correspond to or be directly or indirectly attached to a given feature in the array. In some embodiments, a given feature in the array includes two or more species of oligonucleotides (e.g., capture probes). In some embodiments, the two or more species of oligonucleotides (e.g., capture probes) attached directly or indirectly to a given feature on the array include a common (e.g., identical) spatial barcode.

In some embodiments, an array can include a capture probe attached directly or indirectly to the substrate. The capture probe can include a capture domain (e.g., a nucleotide sequence) that can specifically bind (e.g., hybridize) to a target analyte (e.g., mRNA, DNA, or protein) within a sample. In some embodiments, a substrate includes one or more capture probes that are designed to capture analytes from one or more organisms. The capture probes can be attached to a substrate or feature using a variety of techniques. In some embodiments where the capture probe is immobilized on a feature of the array indirectly, e.g., via hybridization to a surface probe capable of binding the capture probe, the capture probe can further include an upstream sequence (5′ to the sequence that hybridizes to the nucleic acid, e.g., RNA of the tissue sample) that is capable of hybridizing to 5′ end of a surface probe. Alone, the capture domain of the capture probe can be seen as a capture domain oligonucleotide, which can be used in the synthesis of the capture probe in embodiments where the capture probe is immobilized on the array indirectly.

Arrays can be prepared by a variety of methods. In some embodiments, arrays are prepared through the synthesis (e.g., in situ synthesis) of oligonucleotides on the array, or by jet printing or lithography. For example, light-directed synthesis of high-density DNA oligonucleotides can be achieved by photolithography or solid-phase DNA synthesis. To implement photolithographic synthesis, synthetic linkers modified with photochemical protecting groups can be attached to a substrate and the photochemical protecting groups can be modified using a photolithographic mask (applied to specific areas of the substrate) and light, thereby producing an array having localized photodeprotection. Many of these methods are known in the art, and are described e.g., in Miller et al., “Basic concepts of microarrays and potential applications in clinical microbiology.” Clinical Microbiology Reviews 22.4 (2009): 611-633; US201314111482A; U.S. Pat. No. 9,593,365B2; US2019203275; and WO2018091676.

An array for spatial analysis can be generated by various methods as described herein. In some embodiments, the array has a plurality of capture probes comprising spatial barcodes. These spatial barcodes and their relationship to the locations on the array can be determined. In some cases, such information is readily available, because the oligonucleotides are spotted, printed, or synthesized on the array with a predetermined pattern. In some cases, the spatial barcode can be decoded by methods described herein, e.g., by in situ sequencing, by various labels associated with the spatial barcodes etc. In some embodiments, an array can be used as a template to generate a daughter array. Thus, the spatial barcode can be transferred to the daughter array with a known pattern.

In some embodiments, an array includes a plurality of features. In some embodiments, features within an array have an irregular arrangement or relationship to one another, such that no discernable pattern or regularity is evident in the geometrical spacing relationships among the features. For example, features within an array may be positioned randomly with respect to one another. Alternatively, features within an array may be positioned irregularly, but the spacings can be selected deterministically to ensure that the resulting arrangement of features is irregular. In some embodiments, features within an array are positioned regularly with respect to one another to form a pattern. In some embodiments, features within an array are positioned with a degree of regularity with respect to one another such that the array of features is neither perfectly regular nor perfectly irregular (i.e., the array is “partially regular”). In some embodiments, arrays of features can have a variable geometry. In general, arrays of different feature densities can be prepared by adjusting the spacing between adjacent features in the array. In some embodiments, the features can be positioned such that the center-to-center distance or inter-feature distance is less than 100 microns. In some embodiments, the spacing between micro-features can be less than 90 microns (center-to-center distance), less than 80 microns, less than 70 microns, less than 60 microns, less than 50 microns, or any values in between.

An array of features can have any appropriate resolution. In some embodiments, an array of features can have a spatially constant (e.g., within a margin of error) resolution. In some embodiments, depending upon the spatial resolution at which the sample is to be investigated, the sample can be selectively associated with the portion of the array that corresponds approximately to the desired spatial resolution of the measurement. Arrays of spatially varying resolution can be implemented in a variety of ways. In general, the size of the array (which corresponds to the maximum dimension of the smallest boundary that encloses all features in the array along one coordinate direction) can be selected as desired, based on criteria such as the size of the sample, the feature diameter, and the density of capture probes within each feature.

Arrays can be prepared by depositing features (e.g., droplets, beads) on a substrate surface to produce a spatially-barcoded array. Methods of depositing (e.g., droplet manipulation) features are known in the art (see, U.S. Patent Application Publication No. 20080132429, Rubina, A. Y., et al., Biotechniques. 2003 May; 34(5):1008-14, 1016-20, 1022 and Vasiliskov et al. Biotechniques. 1999 September; 27(3):592-4, 596-8).

Large scale commercial manufacturing methods allow for millions of oligonucleotides to be attached to an array. Commercially available arrays include those from Roche NimbleGen, Inc., (Wisconsin) , Affymetrix (ThermoFisher Scientific) and Visium Gene Expression Slides (10× Genomics).

In some embodiments, arrays can be prepared according to the methods set forth in WO 2012140224, WO 2014060483, WO 2016162309, WO 2017019456, WO 2018091676, and WO 2012140224, and U.S. Patent Application No. 20180245142.

Beads

As discussed above, in some embodiments, the devices of the present disclosure can include a plurality of beads. In some embodiments, a bead can be a particle. A bead can be porous, non-porous, solid, semi-solid, andor a combination thereof. In some embodiments, a bead can be dissolvable, disruptable, andor degradable, whereas in certain embodiments, a bead is not degradable. A semi-solid bead can be a liposomal bead. Solid beads can include metals including, without limitation, iron oxide, gold, and silver. In some embodiments, the bead can be a silica bead. In some embodiments, the bead can be rigid. In some embodiments, the bead can be flexible andor compressible.

The bead can be a macromolecule. The bead can be formed of nucleic acid molecules bound together. The bead can be formed via covalent or non-covalent assembly of molecules (e.g., macromolecules), such as monomers or polymers. Polymers or monomers can be natural or synthetic. Polymers or monomers can be or include, for example, nucleic acid molecules (e.g., DNA or RNA).

A bead can be rigid, or flexible andor compressible. A bead can include a coating including one or more polymers. Such a coating can be disruptable or dissolvable. In some embodiments, a bead includes a spectral or optical label (e.g., dye) attached directly or indirectly (e.g., through a linker) to the bead. For example, a bead can be prepared as a colored preparation (e.g., a bead exhibiting a distinct color within the visible spectrum) that can change color (e.g., colorimetric beads) upon application of a desired stimulus (e.g., heat andor chemical reaction) to form differently colored beads (e.g., opaque andor clear beads).

A bead can include natural andor synthetic materials. For example, a bead can include a natural polymer, a synthetic polymer or both natural and synthetic polymers. Examples of natural polymers include, without limitation, proteins, sugars such as deoxyribonucleic acid, rubber, cellulose, starch (e.g., amylose, amylopectin), enzymes, polysaccharides, silks, polyhydroxyalkanoates, chitosan, dextran, collagen, carrageenan, ispaghula, acacia, agar, gelatin, shellac, sterculia gum, xanthan gum, corn sugar gum, guar gum, gum karaya, agarose, alginic acid, alginate, or natural polymers thereof. Examples of synthetic polymers include, without limitation, acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacryl ate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) andor combinations (e.g., co-polymers) thereof. Beads can also be formed from materials other than polymers, including for example, lipids, micelles, ceramics, glass-ceramics, material composites, metals, andor other inorganic materials.

In some embodiments, a bead is a degradable bead. A degradable bead can include one or more species (e.g., disulfide linkers, primers, other oligonucleotides, etc.) with a labile bond such that, when the beadspecies is exposed to the appropriate stimuli, the labile bond is broken and the bead degrades. The labile bond can be a chemical bond (e.g., covalent bond, ionic bond) or can be another type of physical interaction (e.g., van der Waals interactions, dipole-dipole interactions, etc.). In some embodiments, a cross-linker used to generate a bead can include a labile bond. Upon exposure to the appropriate conditions, the labile bond can be broken and the bead degraded. For example, upon exposure of a polyacrylamide gel bead including cystamine cross-linkers to a reducing agent, the disulfide bonds of the cystamine can be broken and the bead degraded. Degradation can refer to the disassociation of a bound or entrained species (e.g., disulfide linkers, primers, other oligonucleotides, etc.) from a bead, both with and without structurally degrading the physical bead itself. For example, entrained species can be released from beads through osmotic pressure differences due to, for example, changing chemical environments. By way of example, alteration of bead pore volumes due to osmotic pressure differences can generally occur without structural degradation of the bead itself. In some embodiments, an increase in pore volume due to osmotic swelling of a bead can permit the release of entrained species within the bead. In some embodiments, osmotic shrinking of a bead can cause a bead to better retain an entrained species due to pore volume contraction.

In some embodiments, a bead can be formed from materials that include degradable chemical cross-linkers, such as N,N′-bis-(acryloyl)cystamine (BAC) or cystamine. Degradation of such degradable cross-linkers can be accomplished through any variety of mechanisms. In some examples, a bead can be contacted with a chemical degrading agent that can induce oxidation, reduction or other chemical changes. For example, a chemical degrading agent can be a reducing agent, such as dithiothreitol (DTT). Additional examples of reducing agents can include (3-mercaptoethanol, (2 S)-2-amino-1,4-dimercaptobutane (dithiobutylamine or DTBA), tris(2-carboxyethyl) phosphine (TCEP), or combinations thereof.

In some embodiments, exposure to an aqueous solution, such as water, can trigger hydrolytic degradation, and thus degradation of the bead. Beads can also be induced to release their contents upon the application of a thermal stimulus. A change in temperature can cause a variety of changes to a bead. For example, heat can cause a solid bead to liquefy. A change in heat can cause melting of a bead such that a portion of the bead degrades. In some embodiments, heat can increase the internal pressure of the bead components such that the bead ruptures or explodes. Heat can also act upon heat-sensitive polymers used as materials to construct beads.

Where degradable beads are used, it can be beneficial to avoid exposing such beads to the stimulus or stimuli that cause such degradation prior to a given time, in order to, for example, avoid premature bead degradation and issues that arise from such degradation, including for example poor flow characteristics and aggregation. By way of example, where beads include reducible cross-linking groups, such as disulfide groups, it will be desirable to avoid contacting such beads with reducing agents, e.g., DTT or other disulfide cleaving reagents. In such embodiments, treatment of the beads described herein will, in some embodiments be provided free of reducing agents, such as DTT. Because reducing agents are often provided in commercial enzyme preparations, it can be desirable to provide reducing agent free (or DTT free) enzyme preparations in treating the beads described herein. Examples of such enzymes include, e.g., polymerase enzyme preparations, reverse transcriptase enzyme preparations, ligase enzyme preparations, as well as many other enzyme preparations that can be used to treat the beads described herein. The terms “reducing agent free” or “DTT free” preparations refer to a preparation having less than about 110th, less than about 150th, or less than about 1100th of the lower ranges for such materials used in degrading the beads. For example, for DTT, the reducing agent free preparation can have less than about 0.01 millimolar (mM), 0.005 mM, 0.001 mM DTT, 0.0005 mM DTT, or less than about 0.0001 mM DTT. In some embodiments, the amount of DTT can be undetectable.

A degradable bead can be useful to more quickly release an attached capture probe (e.g., a nucleic acid molecule, a spatial barcode sequence, andor a primer) from the bead when the appropriate stimulus is applied to the bead as compared to a bead that does not degrade. For example, for a species bound to an inner surface of a porous bead or in the case of an encapsulated species, the species can have greater mobility and accessibility to other species in solution upon degradation of the bead. In some embodiments, a species can also be attached to a degradable bead via a degradable linker (e.g., disulfide linker). The degradable linker can respond to the same stimuli as the degradable bead or the two degradable species can respond to different stimuli. For example, a capture probe having one or more spatial barcodes can be attached, via a disulfide bond, to a polyacrylamide bead including cystamine. Upon exposure of the spatially-barcoded bead to a reducing agent, the bead degrades and the capture probe having the one or more spatial barcode sequences is released upon breakage of both the disulfide linkage between the capture probe and the bead and the disulfide linkages of the cystamine in the bead.

The addition of multiple types of labile bonds to a bead can result in the generation of a bead capable of responding to varied stimuli. Each type of labile bond can be sensitive to an associated stimulus (e.g., chemical stimulus, light, temperature, pH, enzymes, etc.) such that release of reagents attached to a bead via each labile bond can be controlled by the application of the appropriate stimulus. Some non-limiting examples of labile bonds that can be coupled to a precursor or bead include an ester linkage (e.g., cleavable with an acid, a base, or hydroxylamine), a vicinal diol linkage (e.g., cleavable via sodium periodate), a Diels-Alder linkage (e.g., cleavable via heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), or a phosphodiester linkage (e.g., cleavable via a nuclease (e.g., DNAase)). A bond can be cleavable via other nucleic acid molecule targeting enzymes, such as restriction enzymes (e.g., restriction endonucleases). Such functionality can be useful in controlled release of reagents from a bead. In some embodiments, another reagent including a labile bond can be linked to a bead after gel bead formation via, for example, an activated functional group of the bead as described above. In some embodiments, a gel bead including a labile bond is reversible. In some embodiments, a gel bead with a reversible labile bond is used to capture one or more regions of interest of a biological sample. For example, without limitation, a bead including a thermolabile bond can be heated by a light source (e.g., a laser) that causes a change in the gel bead that facilitates capture of a biological sample in contact with the gel bead. Capture probes having one or more spatial barcodes that are releasably, cleavably, or reversibly attached to the beads described herein include capture probes that are released or releasable through cleavage of a linkage between the capture probe and the bead, or that are released through degradation of the underlying bead itself, allowing the capture probes having the one or more spatial barcodes to be accessed or become accessible by other reagents, or both.

Beads can have different physical properties. Physical properties of beads can be used to characterize the beads. Non-limiting examples of physical properties of beads that can differ include volume, shape, circularity, density, symmetry, and hardness. For example, beads can be of different volumes. Beads of different diameters can be obtained by using microfluidic channel networks configured to provide beads of a specific volume (e.g., based on channel sizes, flow rates, etc.). In some embodiments, beads have different hardness values that can be obtained by varying the concentration of polymer used to generate the beads. In some embodiments, a spatial barcode attached to a bead can be made optically detectable using a physical property of the capture probe.

In some embodiments, special types of nanoparticles with more than one distinct physical property can be used to make the beads physically distinguishable. For example, Janus particles with both hydrophilic and hydrophobic surfaces can be used to provide unique physical properties.

A bead can generally be of any suitable shape. Examples of bead shapes include, but are not limited to, spherical, non-spherical, oval, oblong, amorphous, circular, cylindrical, cuboidal, hexagonal, and variations thereof In some embodiments, beads can self-assemble into a monolayer. In some embodiments, the bead can be approximately spherical. In some embodiments, the bead can be approximately cylindrical. In some embodiments, beads can be of uniform size. In some embodiments, beads can be of heterogeneous size.

In some embodiments, the bead can have a diameter or maximum dimension no larger than 100 μm (e.g., no larger than 95 μm, 90 μm, 85 μm, 80 μm, 75 μm, 70 μm, 65 μm, 60 μm, 55 μm, 50 μm, 45 μm, 40 μm, 35 μm, 30 μm, 25 μm, 20 μm, 15 μm, 14 μm, 13 μm, 12 μm, 11 μm, 10 μm, 9 μm, 8 μm, 7 μm, 6 μm, 5 μm, 4 μm, 3 μm, 2 μm, or 1 μm.) In some embodiments, a plurality of beads has an average diameter no larger than 100 μm. In some embodiments, a plurality of beads has an average diameter or maximum dimension no larger than 95 μm, 90 μm, 85 μm, 80 μm, 75 μm, 70 μm, 65 μm, 60 μm, 55 μm, 50 μm, 45 μm, 40 μm, 35 μm, 30 μm, 25 μm, 20 μm, 15 μm, 14 μm, 13 μm, 12 μm, 11 μm, 10 μm, 9 μm, 8 μm, 7 μm, 6 μm, 5 μm, 4 μm, 3 μm, 2 μm, or 1 μm.

In some embodiments, the volume of the bead can be at least about 1 μm³, e.g., at least 1 μm³, 2 μm³, 3 μm³, 4 μm³, 5 μm³, 6 μm³, 7 μm³, 8 μm³, 9 μm³, 10 μm³, 12 μm³, 14 μm³, 16 μm³, 18 μm³, 20 μm³, 25 μm³, 30 μm³, 35 μm³, 40 μm³, 45 μm³, 50 μm³, 55 μm³, 60 μm³, 65 μm³, 70 μm³, 75 μm³, 80 μm³, 85 μm³, 90 μm³, 95 μm³, 100 μm³, 125 μm³, 150 μm³, 175 μm³, 200 μm³, 250 μm³, 300 μm³, 350 μm³, 400 μm³, 450 μm³, 500 μm³, 550 μm³, 600 μm³, 650 μm³, 700 μm³, 750 μm³, 800 μm³, 850 μm³, 900 μm³, 950 μm³, 1000 μm³, 1200 μm³, 1400 μm³, 1600 μm³, 1800 μm³, 2000 μm³, 2200 μm³, 2400 μm³, 2600 μm³, 2800 μm³, 3000 μm³, or greater.

In some embodiments, beads can be of a nanometer scale (e.g., beads can have a diameter or maximum cross-sectional dimension of about 100 nanometers (nm) to about 900 nanometers (nm) (e.g., 850 nm or less, 800 nm or less, 750 nm or less, 700 nm or less, 650 nm or less, 600 nm or less, 550 nm or less, 500 nm or less, 450 nm or less, 400 nm or less, 350 nm or less, 300 nm or less, 250 nm or less, 200 nm or less, 150 nm or less). A plurality of beads can have an average diameter or average maximum cross-sectional dimension of about 100 nanometers (nm) to about 900 nanometers (nm) (e.g., 850 nm or less, 800 nm or less, 750 nm or less, 700 nm or less, 650 nm or less, 600 nm or less, 550 nm or less, 500 nm or less, 450 nm or less, 400 nm or less, 350 nm or less, 300 nm or less, 250 nm or less, 200 nm or less, 150 nm or less). In some embodiments, a bead has a diameter or volume that is about the diameter of a single biological particle (e.g., a single cell, cell bead, or nucleus under evaluation).

In some embodiments, a bead can identify multiple analytes (e.g., nucleic acids, proteins, chromatin, metabolites, drugs, gRNA, and lipids) from a single biological particle (e.g., a single cell, cell bead, or nucleus). In some embodiments, a bead can identify a single analyte from a single cell or nucleus (e.g., mRNA).

A bead can have a tunable pore volume. The pore volume can be chosen to, for instance, retain denatured nucleic acids. The pore volume can be chosen to maintain diffusive permeability to exogenous chemicals such as sodium hydroxide (NaOH) andor endogenous chemicals such as inhibitors. A bead can be formed of a biocompatible andor biochemically compatible material, andor a material that maintains or enhances cell or nuclei viability. A bead can be formed from a material that can be depolymerized thermally, chemically, enzymatically, andor optically.

In some embodiments, beads can be non-covalently loaded with one or more reagents. The beads can be non-covalently loaded by, for instance, subjecting the beads to conditions sufficient to swell the beads, allowing sufficient time for the reagents to diffuse into the interiors of the beads, and subjecting the beads to conditions sufficient to de-swell the beads. Swelling of the beads can be accomplished, for instance, by placing the beads in a thermodynamically favorable solvent, subjecting the beads to a higher or lower temperature, subjecting the beads to a higher or lower ion concentration, andor subjecting the beads to an electric field.

The swelling of the beads can be accomplished by various swelling methods. In some embodiments, swelling is reversible (e.g., by subjecting beads to conditions that promote de-swelling). In some embodiments, the de-swelling of the beads is accomplished, for instance, by transferring the beads in a thermodynamically unfavorable solvent, subjecting the beads to lower or higher temperatures, subjecting the beads to a lower or higher ion concentration, andor adding or removing an electric field. The de-swelling of the beads can be accomplished by various de-swelling methods. In some embodiments, de-swelling is reversible (e.g., subject beads to conditions that promote swelling). In some embodiments, the de-swelling of beads can include transferring the beads to cause pores in the bead to shrink. The shrinking can then hinder reagents within the beads from diffusing out of the interiors of the beads. The hindrance created can be due to steric interactions between the reagents and the interiors of the beads. The transfer can be accomplished microfluidically. For instance, the transfer can be achieved by moving the beads from one co-flowing solvent stream to a different co-flowing solvent stream. The swellability andor pore volume of the beads can be adjusted by changing the polymer composition of the bead.

A bead can include a polymer that is responsive to temperature so that when the bead is heated or cooled, the characteristics or dimensions of the bead can change. For example, a polymer can include poly(N-isopropylacrylamide). A gel bead can include poly(N-isopropylacrylamide) and when heated the gel bead can decrease in one or more dimensions (e.g., a cross-sectional diameter, multiple cross-sectional diameters). A temperature sufficient for changing one or more characteristics of the gel bead can be, for example, at least about 0 degrees Celsius (° C.), 1° C., 2° C., 3° C., 4° C., 5° C., 10° C., or higher. For example, the temperature can be about 4° C. In some embodiments, a temperature sufficient for changing one or more characteristics of the gel bead can be, for example, at least about 25° C., 30° C., 35° C., 37° C., 40° C., 45° C., 50° C., or higher. For example, the temperature can be about 37° C.

Functionalization of beads for attachment of capture probes can be achieved through a wide range of different approaches, including, without limitation, activation of chemical groups within a polymer, incorporation of active or activatable functional groups in the polymer structure, or attachment at the pre-polymer or monomer stage in bead production. The bead can be functionalized to bind to targeted analytes, such as nucleic acids, proteins, carbohydrates, lipids, metabolites, peptides, or other analytes.

In some embodiments, a bead can contain molecular precursors (e.g., monomers or polymers), which can form a polymer network via polymerization of the molecular precursors. In some embodiments, a precursor can be an already polymerized species capable of undergoing further polymerization via, for example, a chemical cross-linkage. In some embodiments, a precursor can include one or more of an acrylamide or a methacrylamide monomer, oligomer, or polymer. In some embodiments, the bead can include prepolymers, which are oligomers capable of further polymerization. For example, polyurethane beads can be prepared using prepolymers. In some embodiments, a bead can contain individual polymers that can be further polymerized together (e.g., to form a co-polymer). In some embodiments, a bead can be generated via polymerization of different precursors, such that they include mixed polymers, co-polymers, andor block co-polymers. In some embodiments, a bead can include covalent or ionic bonds between polymeric precursors (e.g., monomers, oligomers, and linear polymers), nucleic acid molecules (e.g., oligonucleotides), primers, and other entities. In some embodiments, covalent bonds can be carbon-carbon bonds or thioether bonds.

In some embodiments, a bead can include an acrydite moiety, which in certain aspects can be used to attach one or more capture probes to the bead. In some embodiments, an acrydite moiety can refer to an acrydite analogue generated from the reaction of acrydite with one or more species (e.g., disulfide linkers, primers, other oligonucleotides, etc.), such as, without limitation, the reaction of acrydite with other monomers and cross-linkers during a polymerization reaction. Acrydite moieties can be modified to form chemical bonds with a species to be attached, such as a capture probe. Acrydite moieties can be modified with thiol groups capable of forming a disulfide bond or can be modified with groups already including a disulfide bond. The thiol or disulfide (via disulfide exchange) can be used as an anchor point for a species to be attached or another part of the acrydite moiety can be used for attachment. In some embodiments, attachment can be reversible, such that when the disulfide bond is broken (e.g., in the presence of a reducing agent), the attached species is released from the bead. In some embodiments, an acrydite moiety can include a reactive hydroxyl group that can be used for attachment of species.

In some embodiments, precursors (e.g., monomers or cross-linkers) that are polymerized to form a bead can include acrydite moieties, such that when a bead is generated, the bead also includes acrydite moieties. The acrydite moieties can be attached to a nucleic acid molecule (e.g., an oligonucleotide), which can include a priming sequence (e.g., a primer for amplifying target nucleic acids, random primer, primer sequence for messenger RNA) andor one or more capture probes. The one or more capture probes can include sequences that are the same for all capture probes coupled to a given bead andor sequences that are different across all capture probes coupled to the given bead. The capture probe can be incorporated into the bead. In some embodiments, the capture probe can be incorporated or attached to the bead such that the capture probe retains a free 3′ end. In some embodiments, the capture probe can be incorporated or attached to the bead such that the capture probe retains a free 5′ end. In some embodiments, beads can be functionalized such that each bead contains a plurality of different capture probes. For example, a bead can include a plurality of capture probes e.g., Capture Probe 1, Capture Probe 2, and Capture Probe 3, and each of Capture Probes 1, Capture Probes 2, and Capture Probes 3 contain a distinct capture domain (e.g., capture domain of Capture Probe 1 includes a poly(dT) capture domain, capture domain of Capture Probe 2 includes a gene-specific capture domain, and capture domain of Capture Probe 3 includes a CRISPR-specific capture domain). By functionalizing beads to contain a plurality of different capture domains per bead, the level of multiplex capability for analyte detection can be improved.

In some embodiments, precursors (e.g., monomers or cross-linkers) that are polymerized to form a bead can include a functional group that is reactive or capable of being activated such that when it becomes reactive it can be polymerized with other precursors to generate beads including the activated or activatable functional group. The functional group can then be used to attach additional species (e.g., disulfide linkers, primers, other oligonucleotides, etc.) to the beads. For example, some precursors including a carboxylic acid (COOH) group can co-polymerize with other precursors to form a bead that also includes a COOH functional group. In some embodiments, acrylic acid (a species including free COOH groups), acrylamide, and bis(acryloyl)cystamine can be co-polymerized together to generate a bead including free COOH groups. The COOH groups of the bead can be activated (e.g., via 1-Ethyl-3-(3-dimethylaminopropyl)carbodiimide (EDC) and N-Hydroxysuccinimide (NHS) or 4-(4,6-Dimethoxy-1,3,5-triazin-2-yl)-4-methylmorpholinium chloride (DMTMM)) such that they are reactive (e.g., reactive to amine functional groups where EDCNHS or DMTMM are used for activation). The activated COOH groups can then react with an appropriate species (e.g., a species including an amine functional group where the carboxylic acid groups are activated to be reactive with an amine functional group) as a functional group on a moiety to be linked to the bead.

Beads including disulfide linkages in their polymeric network can be functionalized with additional species (e.g., disulfide linkers, primers, other oligonucleotides, etc.) via reduction of some of the disulfide linkages to free thiols. The disulfide linkages can be reduced via, for example, the action of a reducing agent (e.g., DTT, TCEP, etc.) to generate free thiol groups, without dissolution of the bead. Free thiols of the beads can then react with free thiols of a species or a species including another disulfide bond (e.g., via thiol-disulfide exchange) such that the species can be linked to the beads (e.g., via a generated disulfide bond). In some embodiments, free thiols of the beads can react with any other suitable group. For example, free thiols of the beads can react with species including an acrydite moiety. The free thiol groups of the beads can react with the acrydite via Michael addition chemistry, such that the species including the acrydite is linked to the bead. In some embodiments, uncontrolled reactions can be prevented by inclusion of a thiol capping agent such as N-ethylmaleimide or iodoacetate.

Activation of disulfide linkages within a bead can be controlled such that only a small number of disulfide linkages are activated. Control can be exerted, for example, by controlling the concentration of a reducing agent used to generate free thiol groups andor concentration of reagents used to form disulfide bonds in bead polymerization. Controlling the number of disulfide linkages that are reduced to free thiols can be useful in ensuring bead structural integrity during functionalization. In some embodiments, optically-active agents, such as fluorescent dyes can be coupled to beads via free thiol groups of the beads and used to quantify the number of free thiols present in a bead andor track a bead.

In some embodiments, addition of moieties to a bead after bead formation can be advantageous. For example, addition of a capture probe after bead formation can avoid loss of the species (e.g., disulfide linkers, primers, other oligonucleotides, etc.) during chain transfer termination that can occur during polymerization. In some embodiments, smaller precursors (e.g., monomers or cross linkers that do not include side chain groups and linked moieties) can be used for polymerization and can be minimally hindered from growing chain ends due to viscous effects. In some embodiments, functionalization after bead synthesis can minimize exposure of species (e.g., oligonucleotides) to be loaded with potentially damaging agents (e.g., free radicals) andor chemical environments. In some embodiments, the generated hydrogel can possess an upper critical solution temperature (UCST) that can permit temperature driven swelling and collapse of a bead. Such functionality can aid in oligonucleotide (e.g., a primer) infiltration into the bead during subsequent functionalization of the bead with the oligonucleotide. Post-production functionalization can also be useful in controlling loading ratios of species in beads, such that, for example, the variability in loading ratio is minimized. Species loading can also be performed in a batch process such that a plurality of beads can be functionalized with the species in a single batch.

Reagents can be encapsulated in beads during bead generation (e.g., during polymerization of precursors). Such reagents can or cannot participate in polymerization. Such reagents can be entered into polymerization reaction mixtures such that generated beads include the reagents upon bead formation. In some embodiments, such reagents can be added to the beads after formation. Such reagents can include, for example, capture probes (e.g., oligonucleotides), reagents for a nucleic acid amplification reaction (e.g., primers, polymerases, dNTPs, co-factors (e.g., ionic co-factors), buffers) including those described herein, reagents for enzymatic reactions (e.g., enzymes, co-factors, chemical substrates, buffers), reagents for nucleic acid modification reactions such as polymerization, ligation, or digestion, andor reagents for template preparation (e.g., tagmentation) for one or more sequencing platforms (e.g., Nextera® for Illumina®). Such reagents can include one or more enzymes described herein, including without limitation, polymerase, reverse transcriptase, restriction enzymes (e.g., endonuclease), transposase, ligase, proteinase K, DNAse, etc. Such reagents can also or alternatively include one or more reagents such as lysis agents, inhibitors, inactivating agents, chelating agents, stimulus agents. Trapping of such reagents can be controlled by the polymer network density generated during polymerization of precursors, control of ionic charge within the bead (e.g., via ionic species linked to polymerized species), or by the release of other species. Encapsulated reagents can be released from a bead upon bead degradation andor by application of a stimulus capable of releasing the reagents from the bead. In some embodiments, the beads or bead arrangements can be incubated in permeabilization reagents as described herein.

In some embodiments, the beads can also include (e.g., encapsulate or have attached thereto) a plurality of capture probes that include spatial barcodes, and the optical properties of the spatial barcodes can be used for optical detection of the beads. For example, the absorbance of light by the spatial barcodes can be used to distinguish the beads from one another. In some embodiments, a detectable label can directly or indirectly attach to a spatial barcode and provide optical detection of the bead. In some embodiments, each bead in a group of one or more beads has a unique detectable label, and detection of the unique detectable label determines the location of the spatial barcode sequence associated with the bead.

Optical properties giving rise to optical detection of beads can be due to optical properties of the bead surface (e.g., a detectable label attached to the bead), or optical properties from the bulk region of the bead (e.g., a detectable label incorporated during bead formation or an optical property of the bead itself). In some embodiments, a detectable label can be associated with a bead or one or more moieties coupled to the bead.

In some embodiments, the beads include a plurality of detectable labels. For example, a fluorescent dye can be attached to the surface of the beads andor can be incorporated into the beads. Different intensities of the different fluorescent dyes can be used to increase the number of optical combinations that can be used to differentiate between beads. For example, if N is the number of fluorescent dyes (e.g., between 2 and 10 fluorescent dyes, such as 4 fluorescent dyes) and M is the possible intensities for the dyes (e.g., between 2 and 50 intensities, such as 20 intensities), then M^{N} are the possible distinct optical combinations. In one example, 4 fluorescent dyes with 20 possible intensities can be used to generate 160,000 distinct optical combinations.

One or more optical properties of the beads or biological contents, such as cells, cell beads, or nuclei, can be used to distinguish the individual beads or biological contents from other beads or biological contents. In some embodiments, the beads are made optically detectable by including a detectable label having optical properties to distinguish the beads from one another.

In some embodiments, optical properties of the beads can be used for optical detection of the beads. For example, without limitation, optical properties can include absorbance, birefringence, color, fluorescence, luminosity, photosensitivity, reflectivity, refractive index, scattering, or transmittance. For example, beads can have different birefringence values based on degree of polymerization, chain length, or monomer chemistry.

In some embodiments, nanobeads, such as quantum dots or Janus beads, can be used as optical labels or components thereof. For example, a quantum dot can be attached to a spatial barcode of a bead.

Optical labels of beads can provide enhanced spectral resolution to distinguish (e.g., identify) between beads with unique spatial barcodes (e.g., beads including unique spatial barcode sequences). That is, the beads are manufactured in a way that the optical labels and the barcodes on the beads (e.g., spatial barcodes) are correlated with each other. In some aspects, the beads can be loaded into a flowcell such that beads are arrayed in a closely packed manner (e.g., single-cell or single nucleus resolution). Imaging can be performed, and the spatial location of the barcodes can be determined (e.g., based on information from a look-up table (LUT)). The optical labels for spatial profiling allow for quick deconvolution of bead-barcode (e.g., spatial barcode) identify.

In some examples, a lookup table (LUT) can be used to associate a property (e.g., an optical label, such as a color andor intensity) of the bead with the barcode sequence. The property may derive from the particle (e.g., bead) or an optical label associated with the bead. The beads can be imaged to obtain optical information of the bead, including, for example, the property (e.g., color andor intensity) of the bead or the optical label associated with the bead, and optical information of the biological sample. For example, an image can include optical information in the visible spectrum, non-visible spectrum, or both. In some embodiments, multiple images can be obtained across various optical frequencies.

In some embodiments, the optical label has a characteristic electromagnetic spectrum. As used herein, the “electromagnetic spectrum” refers to the range of frequencies of electromagnetic radiation. In some embodiments, the optical label has a characteristic absorption spectrum. As used herein, the “absorption spectrum” refers to the range of frequencies of electromagnetic radiation that are absorbed. The “electromagnetic spectrum” or “absorption spectrum” can lead to different characteristic spectrum. In some embodiments, the peak radiation or the peak absorption occurs at 380-450 nm (Violet), 450-485 nm (Blue), 485-500 nm (Cyan), 500-565 nm (Green), 565-590 nm (Yellow), 590-625 nm (Orange), or 625-740 nm (Red). In some embodiments, the peak radiation or the peak absorption occurs around 400 nm, 460 nm, or 520 nm.

In one aspect, the methods, compositions, devices, systems, and kits of the present disclosure may be used to magnetically manipulate target features on a substrate, wherein the target features are cell beads. A cell bead can be a biological particle andor one or more of its macromolecular constituents encased inside of a gel or polymer matrix, such as via polymerization of a droplet containing the biological particle and precursors capable of being polymerized or gelled. A cell bead can contain biological particles (e.g., a cell) or macromolecular constituents (e.g., RNA, DNA, proteins, etc.) of the biological particles. A cell bead may include a single cell or multiple cells, or a derivative of the single cell or multiple cells. For example after lysing and washing the cells, inhibitory components from cell lysates can be washed away and the macromolecular constituents can be bound as cell beads. Cell beads may be or include a cell, cell derivative, cellular material andor material derived from the cell in, within, or encased in a matrix, such as a polymeric matrix. In some cases, a cell bead may comprise a live cell. In some instances, the live cell may be capable of being cultured when enclosed in a gel or polymer matrix, or of being cultured when comprising a gel or polymer matrix. In some instances, the polymer or gel may be diffusively permeable to certain components and diffusively impermeable to other components (e.g., macromolecular constituents). Encapsulated biological particles can provide certain potential advantages of being more storable and more portable, and, in some cases, it may be desirable to allow biological particles to incubate for a select period of time before analysis, such as in order to characterize changes in such biological particles over time, either in the presence or absence of different stimuli (or reagents). For a description of methods, compositions, devices, and systems for generating encapsulated cells (e.g., “cell beads”), see, e.g., U.S. Pat. Nos. 10,428,326 and 10,590,244, which are each incorporated by reference in their entirety. In addition, the cell beads may be exposed to an appropriate stimulus to release the biological particles (e.g., cells) or their contents. For example, in some cases, a chemical stimulus may be provided to allow for the degradation of the cell bead and release of the cell or its contents.

In some embodiments, the beads are only deposited to areas of interest (e.g., specific locations on the substrate, specific cell types, and specific tissue structures). Thus, the deposited beads do not necessarily cover the entire biological sample. In some embodiments, one or more regions of a substrate can be masked or modified (e.g., capped capture domains) such that the masked regions do not interact with a corresponding region of the biological sample.

Optical labels can be included while generating the beads. For example, optical labels can be included in the polymer structure of a gel bead, or attached at the pre-polymer or monomer stage in bead production. Optical labels included on the beads can identify the associated spatial barcode on the bead. In some embodiments, the beads include moieties that attach to one or more optical labels (e.g., at a surface of a bead andor within a bead). In some embodiments, optical labels can be loaded into the beads with one or more reagents. For example, reagents and optical labels can be loaded into the beads by diffusion of the reagents (e.g., a solution of reagents including the optical labels). In some embodiments, optical labels can be included while preparing spatial barcodes. For example, spatial barcodes can be prepared by synthesizing molecules including barcode sequences (e.g., using a split pool or combinatorial approach). Optical labels can be attached to spatial barcodes prior to attaching the spatial barcodes to a bead. In some embodiments, optical labels can be included after attaching spatial barcodes to a bead. For example, optical labels can be attached to spatial barcodes coupled to the bead. In some embodiments, spatial barcodes or sequences thereof can be releasably or cleavably attached to the bead. Optical labels can be releasably or non-releasably attached to the bead.

In some embodiments, beads can be affixed or attached to a substrate using photochemical methods. For example, a bead can be functionalized with perfluorophenylazide silane (PFPA silane), contacted with a substrate, and then exposed to irradiation (see, e.g., Liu et al. (2006) Journal of the American Chemical Society 128, 14067-14072). For example, immobilization of antraquinone-functionalized substrates (see, e.g., Koch et al. (2000) Bioconjugate Chem. 11, 474-483).

In some embodiments, beads can be affixed or attached to a substrate covalently, non-covalently, with adhesive, or a combination thereof. The attached beads can be, for example, layered in a monolayer, a bilayer, a trilayer, or as a cluster. As defined herein, a “monolayer” generally refers to an arrayed series of probes, beads, spots, dots, features, micro-locations, or islands that are affixed or attached to a substrate, such that the beads are arranged as one layer of single beads. In some embodiments, the beads are closely packed.

In some embodiments, the monolayer of beads is a located in a predefined area on the substrate. For example, the predefined area can be partitioned with physical barriers, a photomask, divots in the substrate, or wells in the substrate.

In some embodiments, a reactive element is bound directly to a bead. As used herein, the term “reactive element” generally refers to a molecule or molecular moiety that can react with another molecule or molecular moiety to form a covalent bond. Reactive elements include, for example, amines, aldehydes, alkynes, azides, thiols, haloacetyls, pyridyl disulfides, hydrazides, carboxylic acids, alkoxyamines, sulfhydryls, maleimides, Michael acceptors, hydroxyls, and active esters. Some reactive elements, for example, carboxylic acids, can be treated with one or more activating agents (e.g., acylating agents, isourea-forming agents) to increase susceptibility of the reactive element to nucleophilic attack. Non-limiting examples of activating agents include N-hydroxysuccinimide, N-hydroxysulfosuccinimide, 1-ethyl-3-(3-dimethylaminopropyl)carb odiimide, dicyclohexylcarbodiimide, diisopropylcarbodiiimide, 1-hydroxybenzotriazole, (benzotriazol-1-yloxy)tripyrrolidinophosphonium hexfluorophosphate, (benzotriaz01-1-yl)-N,N,N′,N′-tetramethyluronium hexafluorophosphate, 4-(N,N-dimethylamino)pyridine, and carbonyldiimidazole.

For example, hydrogel beads can be treated with an acrylic acid monomer to form acrylic acid-functionalized hydrogel beads. In some cases, the reactive element is bound indirectly to the bead via one or more linkers. As used herein, a “linker” generally refers to a multifunctional (e.g., bifunctional, trifunctional) reagent used for conjugating two or more chemical moieties. A linker can be a cleavable linker that can undergo induced dissociation. For example, the dissociation can be induced by a solvent (e.g., hydrolysis and solvolysis); by irradiation (e.g., photolysis); by an enzyme (e.g., enzymolysis); or by treatment with a solution of specific pH (e.g., pH 4, 5, 6, 7, or 8).

In some embodiments, the reactive element is bound directly to a substrate. For example, a glass slide can be coated with (3-aminopropyl)triethoxysilane. In some embodiments, the reactive element is bound indirectly to a substrate via one or more linkers.

Bead Arrays

“Bead array” refers to an array that includes a plurality of beads as the features in the array. In some embodiments, two or more beads are dispersed onto a substrate to create an array, where each bead is a feature on the array. In some embodiments, the beads are attached to a substrate. For example, the beads can optionally attach to a substrate such as a microscope slide and in proximity to a biological sample (e.g., a tissue section that includes cellsnuclei). The beads can also be suspended in a solution and deposited on a surface (e.g., a membrane, a tissue section, or a substrate (e.g., a microscope slide)). Beads can optionally be dispersed into wells on a substrate, e.g., such that only a single bead is accommodated per well.

Examples of arrays of beads on or within a substrate include beads located in wells such as the BeadChip array (available from Illumina Inc., San Diego, Calif.), arrays used in sequencing platforms from 454 LifeSciences (a subsidiary of Roche, Basel, Switzerland), and array used in sequencing platforms from Ion Torrent (a subsidiary of Life Technologies, Carlsbad, Calif.). Examples of bead arrays are described in, e.g., U.S. Pat. Nos. 6,266,459; 6,355,431; 6,770,441; 6,859,570; 6,210,891; 6,258,568; and 6,274,320; U.S. Pat. Application Publication Nos. 20090026082; 20090127589; 20100137143; 20190177777; and 20100282617; and PCT Patent Application Publication Nos. WO 00063437 and WO 2016162309.

In some embodiments, the bead array includes a plurality of beads. For example, the bead array can include at least 10,000 beads (e.g., at least 100,000 beads, at least 1,000,000 beads, at least 5,000,000 beads, at least 10,000,000 beads). In some embodiments, the plurality of beads includes a single type of bead (e.g., substantially uniform in volume, shape, and other physical properties, such as translucence). In some embodiments, the plurality of beads includes two or more types of different beads.

Bead arrays can be generated by attaching beads (e.g., barcoded beads) to a substrate in a regular pattern, or an irregular arrangement. In some embodiments, the barcode sequences are known before attaching them to the substrate. In some embodiments, the barcode sequences are not known before attaching them to the substrate. Beads can be attached to selective regions on a substrate by, e.g., selectively activating regions on the substrate to allow for attachment of the beads. Activating selective regions on the substrate can include activating or degrading a coating (e.g., a conditionally removable coating as described herein) at the selective regions where the coating has been applied on the substrate, rendering the selective regions more permissive to bead attachment as compared to regions outside of the selected regions. The regions that are rendered more permissive for bead attachment can be configured to fit only one bead or multiple beads (e.g., limited by well size or surface patterning, such as fabrication techniques). Beads bound to the selected regions can form a two-dimensional array on the substrate. The substrate can be uniformly or non-uniformly coated with the coating. The beads can be any suitable beads described herein, including beads that are attached to one or more spatial barcodes. Beads can be attached to the selected regions according to any of the methods suitable for attaching beads to substrates described herein, such as through covalent bonds, non-covalent bonds, or chemical linkers.

Any variety of suitable patterning techniques can be used to attach beads to a substrate surface. In some embodiments, in a non-limiting way, physical techniques such as inkjet printing, optical and optoelectronic cell trapping, laser-based patterning, acoustic patterning, dielectrophoresis, or magnetic techniques can be used to pattern the substrate. Alternatively, chemical andor physio-chemical techniques can be used such as, in a non-limiting way, surface chemistry methods, microcontact printing, microwells and filtration, DUV patterning, or patterning in microfluidic devices combined with microcontact printing (See, e.g., Martinez-Rivas, A., Methods of micropatterning and manipulation of cells for biomedical applications, Micromachines (Basel) 8, (2017)).

The coating can be photoreactive, and selectively activating or degrading the coating involves exposing selected regions of the coating to light or radiation. Selectivity can be achieved through the application of photomasks. Regions of the coating that are exposed to light can be rendered more permissive for bead attachment (e.g., more adhesive), as compared to regions not exposed to light (e.g., regions protected from the light by a photomask). When applied to the substrate, the beads thus preferentially attach to the more permissive regions on the substrate, and unattached beads can optionally be removed from the substrate. The light source andor the photomask can be adjusted to allow further sites on the substrate to become more permissive for bead attachment, allowing additional beads to be attached at these sites. Alternatively, a different light source, or a different photomask can be applied. The process of photopatterning thus allows beads to be attached at predetermined locations on the substrate, thereby generating a bead array.

Beads can be attached iteratively, e.g., a subset of the beads can be attached at one time, and the process can be repeated to attach one or more additional subsets of beads. In some embodiments, the size of the activated spot (e.g., spot on the substrate) is smaller than the size of a bead. For example, a bead can be attached to the activated substrate (e.g., spot) such that only a single bead attaches to the activated substrate. In some embodiments, the substrate can be washed to remove unbound beads. In some embodiments, the substrate can be activated in a second location and a second bead can be attached to the activated substrate surface. This process can be done iteratively to attach beads to the entire substrate, or a portion thereof.

Alternatively, beads can be attached to the substrate all in one step. Furthermore, methods of attaching beads to a substrate are known in the art. Any suitable method can be used, including, in a non-limiting way, specific chemical bonds, non-specific chemical bonds, linkers, physically trapping the beads (e.g., polymer, hydrogel), or any of the methods described herein.

An exemplary workflow for generating a bead array can include selectively rendering a first set of one or more selected regions on a coated substrate to be more permissive for bead attachment as compared to regions outside of the selected regions, applying a plurality of beads to the array and allowing the beads to attach to the first set of selected regions, optionally removing unattached beads, rendering a second set of one or more selected regions more permissive to bead attachment as compared to regions outside the second set of selected regions, applying a plurality of beads and allowing the beads to attach to the second set of selected regions, and optionally removing the unattached beads. This iterative process can be carried out for any number of times to generate a patterned bead array.

Another exemplary process includes activating a first region on a coated substrate and exposing the activated first region to a plurality of barcoded beads, so that a first set of one or more beads are bound to the first region; and activating a second region on the coated substrate and exposing the activated second region to a plurality of barcoded beads, so that a second set of one or more beads are bound to the second region, wherein the first set of one or more beads comprise an identical first oligonucleotide sequence unique to the first region of the surface of the substrate, and the second set of one or more beads comprise an identical second oligonucleotide sequence unique to the second region of the surface of the substrate, and wherein the first and second oligonucleotide sequences are different. Additional regions on the coated substrate may be activated and exposed to additional barcoded beads. Each set of barcoded beads can include an oligonucleotide sequence that is different from all other sets of barcoded beads and that is unique to the location of the activated region. Additionally, the first set of one or more beads and the second set of one or more beads can be different. In other words, the first set of one or more beads and the second set of one or more beads can have different surface chemistries, different compositions (e.g., solid bead, gel bead, silica bead) (e.g., nanoparticles vs microparticles), andor physical volumes. In some embodiments, a third set of one or more beads, a fourth set of one or more beads, a fifth set of one or more beads or more can have different surface chemistries, different compositions (e.g., solid bead, gel bead, silica bead)(e.g., nanoparticles vs microparticles), andor physical volumes can be attached to the substrate surface. The methods can include removing the beads that do not bind to the first, second, andor any of the additional regions. In some embodiments, removing the beads comprise washing the beads off the surface of the substrate. The removing may be carried out after each round of or after several rounds of activating a region (e.g., first, second or additional regions on the surface of the substrate), and binding of beads to the activated region. In some instances, each bead is bound to the substrate at a single location. The beads bound to the first, second, and additional regions can form a two-dimensional array of beads on the substrate.

Beads can be attached to selective regions on a substrate by selectively crosslinking beads to a coating that has been applied on the substrate. For example, a plurality of beads can be applied to a substrate having a photocrosslinkable coating, and upon crosslinking of a subset of the beads to the coating, the non-cross-linked beads can be removed, leaving only the cross-linked beads on the substrate. The process can be repeated multiple times.

In some embodiments, a bead array is formed when beads are embedded in a hydrogel layer where the hydrogel polymerizes and secures the relative bead positions. The bead-arrays can be pre-equilibrated and combined with reaction buffers and enzymes (e.g., reverse-transcription mix). In some embodiments, the bead arrays can be stored (e.g., frozen) long-term (e.g., days) prior to use.

Coatings

A surface of a substrate can be coated with a various substances. The coating can comprise a polymer, and activating selected regions on the substrate include modifying the polymer at the respective regions. Modifying the polymer includes, for example, photochemically modifying the polymer by exposing the polymer to radiation or light. Alternatively or additionally, modifying the polymer can include chemically modifying the polymer by contacting the polymer with one or more chemical reagents. In some instances, the coating is a hydrogel. In some instances, the coating comprises a photoreactive polymer. Exemplary photo-reactive polymers include poly(ethylene glycol) (PEG)-based polymers, poly(L-lysine) (PLL)-based polymer, copolymer comprising functionalized or unfunctionalized units of PEG and PLL (e.g., poly-L-lysine-grafted-polyethylene glycol (PLL-g-PEG)), and methacrylated gelatin (GelMA) polymers.

A photoreactive coating can comprise a plurality of photoreactive molecules, which can undergo a chemical reaction (e.g., hydrolysis, oxidation, photolysis) when exposed to light of certain wavelengths or range of wavelengths. A photo-reactive molecule can become reactive when exposed to light and can react with other molecules and form chemical bonds with other molecules.

The coating can include a photo-crosslinkable polymer precursor. A “photo-crosslinkable polymer precursor” refers to a compound that cross-links andor polymerizes upon exposure to light. Exemplary photo-crosslinkable polymers are described, e.g., in Shirai, Polymer Journal 46:859-865 (2014), Ravve, Photocrosslinkable Polymers, Light-Associated Reactions of Synthetic Polymers. Springer, New York, N.Y. (2006), and Ferreira et al. Photocrosslinkable Polymers for Biomedical Applications, Biomedical Engineering—Frontiers and Challenges, Prof. Reza Fazel (Ed.), ISBN: 978-953-307-309-5 (2011). In some embodiments, one or more photoinitiators may also be included to induce andor promote polymerization andor cross-linking. See, e.g., Choi et al. Biotechniques. 2019 Jan; 66(1):40-53, which is incorporated herein by reference in its entirety. Non-limiting examples of photo-crosslinkable polymer precursors include polyethylene (glycol) diacrylate (PEGDA), gelatin-methacryloyl (GelMA), and methacrylated hyaluronic acid (MeHA). In some embodiments, a photo-crosslinkable polymer precursor comprises polyethylene (glycol) diacrylate (PEGDA), gelatin-methacryloyl (GelMA), methacrylated hyaluronic acid (MeHA), or a combination thereof In some embodiments, a photo-crosslinkable polymer precursor (e.g., PAZAM) can be covalently linked (e.g., cross-linked) to a substrate. In some embodiments, a photo-crosslinkable polymer precursor is not covalently linked to a substrate surface. For example, a silane-free acrylamide can be used (See U.S. Patent Application Publication No. 20110059865). The photo-crosslinkable polymer precursor in a feature (e.g., droplet or bead) can be polymerized by any known method. The oligonucleotides can be polymerized in a cross-linked gel matrix (e.g., copolymerized or simultaneously polymerized). In some embodiments, the features containing the photo-crosslinkable polymer precursor deposited on the substrate surface can be exposed to UV light. The UV light can induce polymerization of the photo-crosslinkable polymer precursor and result in the features becoming a gel matrix (e.g., gel pads) on the substrate surface (e.g., array).

Suitable light sources for activating, degrading or crosslinking the coating as described herein include, but are not limited to, Ultraviolet (UV) light (e.g., 250-350 nm or 350-460 nmUV light) and visible light (e.g., broad spectrum visible light). A Digital Micromirror Device (DMD) can also be used to provide the light source.

In some embodiments, a substrate is coated with a surface treatment such as poly(L)-lysine. Additionally or alternatively, the substrate can be treated by silanation, e.g., with epoxy-silane, amino-silane, andor by a treatment with polyacrylamide.

In some embodiments, a substrate is treated to minimize or reduce non-specific analyte hybridization within or between features. For example, treatment can include coating the substrate with a hydrogel, film, andor membrane that creates a physical barrier to non-specific hybridization. Any suitable hydrogel can be used. For example, hydrogel matrices prepared according to the methods set forth in U.S. Pat. Nos. 6,391,937, 9,512,422, and 9,889,422, and U.S. Patent Application Publication Nos. U.S. 20170253918 and U.S. 20180052081, can be used. Treatment can include adding a functional group that is reactive or capable of being activated such that it becomes reactive after application of a stimulus (e.g., photoreactive functional groups). Treatment can include treating with polymers having one or more physical properties (e.g., mechanical, electrical, magnetic, andor thermal) that minimize non-specific binding (e.g., that activate a substrate at certain locations to allow analyte hybridization at those locations).

In some embodiments, the coating is a “conditionally removable coating.” A conditionally removable coating is a coating that can be removed from the surface of a substrate upon application of a releasing agent. In some embodiments, a conditionally removable coating includes a hydrogel as described herein, e.g., a hydrogel including a polypeptide-based material. Non-limiting examples of a hydrogel featuring a polypeptide-based material include a synthetic peptide-based material featuring a combination of spider silk and a trans-membrane segment of human muscle L-type calcium channel (e.g., PEPGEL®), an amphiphilic 16 residue peptide containing a repeating arginine-alanine-aspartate-alanine sequence (RADARADARADARADA) (e.g., PURAMATRIX®), EAK16 (AEAEAKAKAEAEAKAK), KLD12 (KLDLKLDLKLDL), and PGMATRIX™.

In some embodiments, the hydrogel in the conditionally removable coating is a stimulus-responsive hydrogel. A stimulus-responsive hydrogel can undergo a gel-to-solution andor gel-to-solid transition upon application of one or more external triggers (e.g., a releasing agent). See, e.g., Willner, Acc. Chem. Res. 50:657-658, 2017. Non-limiting examples of a stimulus-responsive hydrogel include a thermoresponsive hydrogel, a pH-responsive hydrogel, a light-responsive hydrogel, a redox-responsive hydrogel, an analyte-responsive hydrogel, or a combination thereof In some embodiments, a stimulus-responsive hydrogel can be a multi-stimuli-responsive hydrogel.

A “releasing agent” or “external trigger” is an agent that allows for the removal of a conditionally removable coating from a substrate when the releasing agent is applied to the conditionally removable coating. An external trigger or releasing agent can include physical triggers such as thermal, magnetic, ultrasonic, electrochemical, andor light stimuli as well as chemical triggers such as pH, redox reactions, supramolecular complexes, andor biocatalytically driven reactions. See e.g., Echeverria, et al., Gels (2018), 4, 54; doi:10.3390ge1s4020054. The type of “releasing agent” or “external trigger” can depend on the type of conditionally removable coating. For example, a conditionally removable coating featuring a redox-responsive hydrogel can be removed upon application of a releasing agent that includes a reducing agent such as dithiothreitol (DTT). As another example, a pH-responsive hydrogel can be removed upon the application of a releasing agent that changes the pH.

In some embodiments, a conductive substrate (e.g., a glass slide) is coated with a substance or otherwise modified to confer conductive properties to the glass. In some embodiments, a glass slide can be coated with a conductive coating. In some embodiments, a conductive coating includes tin oxide (TO) or indium tin oxide (ITO). In some embodiments, a conductive coating includes a transparent conductive oxide (TCO). In some embodiments, a conductive coating includes aluminum doped zinc oxide (AZO). In some embodiments, a conductive coating includes fluorine doped tin oxide (FTO).

Capture Probe

A “capture probe” refers to any molecule capable of capturing (directly or indirectly) andor labelling an analyte (e.g., an analyte of interest) in a biological sample. Capture probes can include ribonucleotides andor deoxyribonucleotides as well as synthetic nucleotide residues that are capable of participating in Watson-Crick type or analogous base pair interactions.

In some embodiments, the capture probe is a nucleic acid or a polypeptide. In some embodiments, the capture probe is a conjugate (e.g., an oligonucleotide-antibody conjugate). In some embodiments, the capture probe includes a barcode (e.g., a spatial barcode andor a unique molecular identifier (UMI)) and a capture domain.

In some embodiments, the capture probe can be optionally coupled to a feature by a cleavage domain, such as a disulfide linker. The capture probe can include functional sequences that are useful for subsequent processing, such as functional sequence, which can include a sequencer specific flow cell attachment sequence, e.g., a P5 sequence andor a P7 sequence, as well as functional sequence, which can include sequencing primer sequences, e.g., a R1 primer binding site andor a R2 primer binding site.

In some embodiments, a capture probe includes a capture domain having a sequence that is capable of binding to mRNA andor genomic DNA. For example, the capture probe can include a capture domain that includes a nucleic acid sequence (e.g., a poly(T) sequence) capable of binding to a poly(A) tail of an mRNA andor to a poly(A) homopolymeric sequence present in genomic DNA. In some embodiments, a homopolymeric sequence is added to an mRNA molecule or a genomic DNA molecule using a terminal transferase enzyme in order to produce an analyte that has a poly(A) or poly(T) sequence. For example, a poly(A) sequence can be added to an analyte (e.g., a fragment of genomic DNA) thereby making the analyte capable of capture by a poly(T) capture domain.

Each capture probe can optionally include at least one cleavage domain. The cleavage domain represents the portion of the probe that is used to reversibly attach the probe to an array feature. Further, one or more segments or regions of the capture probe can optionally be released from the array feature by cleavage of the cleavage domain. As an example, spatial barcodes andor universal molecular identifiers (UMIs) can be released by cleavage of the cleavage domain.

In some embodiments, a cleavage domain is absent from the capture probe. Examples of substrates with attached capture probes lacking a cleavage domain are described for example in Macosko et al., (2015) Cell 161, 1202-1214.

Each capture probe can optionally include at least one functional domain. Each functional domain generally includes a functional nucleotide sequence for a downstream analytical step in the overall analysis procedure.

Spatial Barcode

A “spatial barcode” is a contiguous nucleic acid segment or two or more non-contiguous nucleic acid segments that function as a label or identifier that conveys or is capable of conveying spatial information. In some embodiments, a capture probe includes a spatial barcode that possesses a spatial aspect, where the barcode is associated with a particular location within an array or a particular location on a substrate.

A spatial barcode can be included within the capture probe for use in barcoding the target analyte. The functional sequences can generally be selected for compatibility with any of a variety of different sequencing systems, e.g., 454 Sequencing, Ion Torrent Proton or PGM, Illumina X10, PacBio, Nanopore, etc., and the requirements thereof. In some embodiments, functional sequences can be selected for compatibility with non-commercialized sequencing systems. Further, in some embodiments, functional sequences can be selected for compatibility with other sequencing systems, including non-commercialized sequencing systems.

In some embodiments, the spatial barcode can be common to all of the probes attached to a given feature. The spatial barcode can also include a capture domain to facilitate capture of a target analyte.

A spatial barcode can be part of an analyte, or independent from an analyte (e.g., part of the capture probe). A spatial barcode can be a tag attached to an analyte (e.g., a nucleic acid molecule) or a combination of a tag in addition to an endogenous characteristic of the analyte (e.g., size of the analyte or end sequence(s)).

A spatial barcode can be unique. In some embodiments where the spatial barcode is unique, the spatial barcode functions both as a spatial barcode and as a unique molecular identifier (UMI), associated with one particular capture probe.

Spatial barcodes can have a variety of different formats. For example, spatial barcodes can include polynucleotide spatial barcodes; random nucleic acid andor amino acid sequences; and synthetic nucleic acid andor amino acid sequences. In some embodiments, a spatial barcode is attached to an analyte in a reversible or irreversible manner. In some embodiments, a spatial barcode is added to, for example, a fragment of a DNA or RNA sample before, during, andor after sequencing of the sample. In some embodiments, a spatial barcode allows for identification andor quantification of individual sequencing-reads. In some embodiments, a spatial barcode is a used as a fluorescent barcode for which fluorescently labeled oligonucleotide probes hybridize to the spatial barcode.

In some embodiments, the spatial barcode is a nucleic acid sequence that does not substantially hybridize to analyte nucleic acid molecules in a biological sample. In some embodiments, the spatial barcode has less than 80% sequence identity (e.g., less than 70%, 60%, 50%, or less than 40% sequence identity) to the nucleic acid sequences across a substantial part (e.g., 80% or more) of the nucleic acid molecules in the biological sample.

For multiple capture probes that are attached to a common array feature, the one or more spatial barcode sequences of the multiple capture probes can include sequences that are the same for all capture probes coupled to the feature, andor sequences that are different across all capture probes coupled to the feature.

Capture probes attached to a single array feature can include identical (or common) spatial barcode sequences, different spatial barcode sequences, or a combination of both. Capture probes attached to a feature can include multiple sets of capture probes. Capture probes of a given set can include identical spatial barcode sequences. The identical spatial barcode sequences can be different from spatial barcode sequences of capture probes of another set.

The plurality of capture probes can include spatial barcode sequences (e.g., nucleic acid barcode sequences) that are associated with specific locations on a spatial array. For example, a first plurality of capture probes can be associated with a first region, based on a spatial barcode sequence common to the capture probes within the first region, and a second plurality of capture probes can be associated with a second region, based on a spatial barcode sequence common to the capture probes within the second region. The second region may or may not be associated with the first region. Additional pluralities of capture probes can be associated with spatial barcode sequences common to the capture probes within other regions. In some embodiments, the spatial barcode sequences can be the same across a plurality of capture probe molecules.

In some embodiments, multiple different spatial barcodes are incorporated into a single arrayed capture probe. For example, a mixed but known set of spatial barcode sequences can provide a stronger address or attribution of the spatial barcodes to a given spot or location, by providing duplicate or independent confirmation of the identity of the location. In some embodiments, the multiple spatial barcodes represent increasing specificity of the location of the particular array point.

Capture Domain

A capture probe can include at least one capture domain. The “capture domain” can be an oligonucleotide, a polypeptide, a small molecule, or any combination thereof, that binds specifically to a desired analyte. In some embodiments, a capture domain can be used to capture or detect a desired analyte.

In some embodiments, the capture domain is a functional nucleic acid sequence configured to interact with one or more analytes, such as one or more different types of nucleic acids (e.g., RNA molecules and DNA molecules). In some embodiments, the functional nucleic acid sequence can include an N-mer sequence (e.g., a random N-mer sequence), which N-mer sequences are configured to interact with a plurality of DNA molecules. In some embodiments, the functional sequence can include a poly(T) sequence, which poly(T) sequences are configured to interact with messenger RNA (mRNA) molecules via the poly(A) tail of an mRNA transcript. In some embodiments, the functional nucleic acid sequence is the binding target of a protein (e.g., a transcription factor, a DNA binding protein, or a RNA binding protein), where the analyte of interest is a protein.

In some embodiments, the capture domain is capable of priming a reverse transcription reaction to generate cDNA that is complementary to the captured RNA molecules. In some embodiments, the capture domain of the capture probe can prime a DNA extension (polymerase) reaction to generate DNA that is complementary to the captured DNA molecules. In some embodiments, the capture domain can template a ligation reaction between the captured DNA molecules and a surface probe that is directly or indirectly immobilized on the substrate. In some embodiments, the capture domain can be ligated to one strand of the captured DNA molecules. For example, SplintR ligase along with RNA or DNA sequences (e.g., degenerate RNA) can be used to ligate a single-stranded DNA or RNA to the capture domain. In some embodiments, ligases with RNA-templated ligase activity, e.g., SplintR ligase, T4 RNA ligase 2 or KOD ligase, can be used to ligate a single-stranded DNA or RNA to the capture domain. In some embodiments, a capture domain includes a splint oligonucleotide. In some embodiments, a capture domain captures a splint oligonucleotide.

In some embodiments, the capture domain is located at the 3′ end of the capture probe and includes a free 3′ end that can be extended, e.g., by template dependent polymerization, to form an extended capture probe as described herein. In some embodiments, the capture domain includes a nucleotide sequence that is capable of hybridizing to nucleic acid, e.g., RNA or other analyte, present in the cells, cell beads, or nuclei of the biological sample contacted with the array. In some embodiments, the capture domain can be selected or designed to bind selectively or specifically to a target nucleic acid. For example, the capture domain can be selected or designed to capture mRNA by way of hybridization to the mRNA poly(A) tail. Thus, in some embodiments, the capture domain includes a poly(T) DNA oligonucleotide, e.g., a series of consecutive deoxythymidine residues linked by phosphodiester bonds, which is capable of hybridizing to the poly(A) tail of mRNA. In some embodiments, the capture domain can include nucleotides that are functionally or structurally analogous to a poly(T) tail. For example, a poly(U) oligonucleotide or an oligonucleotide included of deoxythymidine analogues. In some embodiments, the capture domain includes at least 10, 11, 12, 13, 14, 15, 16, 17, 18, 19, or 20 nucleotides. In some embodiments, the capture domain includes at least 25, 30, or 35 nucleotides.

In some embodiments, random sequences, e.g., random hexamers or similar sequences, can be used to form all or a part of the capture domain. For example, random sequences can be used in conjunction with poly(T) (or poly(T) analogue) sequences. Thus, where a capture domain includes a poly(T) (or a “poly(T)-like”) oligonucleotide, it can also include a random oligonucleotide sequence (e.g., “poly(T)-random sequence” probe). This can, for example, be located 5′ or 3′ of the poly(T) sequence, e.g., at the 3′ end of the capture domain. The poly(T)-random sequence probe can facilitate the capture of the mRNA poly(A) tail. In some embodiments, the capture domain can be an entirely random sequence. In some embodiments, degenerate capture domains can be used.

The capture domain can be based on a particular gene sequence or particular motif sequence or commonconserved sequence, that it is designed to capture (i.e., a sequence-specific capture domain). Thus, in some embodiments, the capture domain is capable of binding selectively to a desired sub-type or subset of nucleic acid, for example a particular type of RNA, such as mRNA, rRNA, tRNA, SRP RNA, tmRNA, snRNA, snoRNA, SmY RNA, scaRNA, gRNA, RNase P, RNase MRP, TERC, SL RNA, aRNA, cis-NAT, crRNA, lncRNA, miRNA, piRNA, siRNA, shRNA, tasiRNA, rasiRNA, 7SK, eRNA, ncRNA or other types of RNA. In a non-limiting example, the capture domain can be capable of binding selectively to a desired subset of ribonucleic acids, for example, microbiome RNA, such as 16S rRNA.

In some embodiments, a capture domain includes an “anchor” or “anchoring sequence”, which is a sequence of nucleotides that is designed to ensure that the capture domain hybridizes to the intended analyte. In some embodiments, an anchor sequence includes a sequence of nucleotides, including a 1-mer, 2-mer, 3-mer or longer sequence. In some embodiments, the short sequence is random. For example, a capture domain including a poly(T) sequence can be designed to capture an mRNA. In such embodiments, an anchoring sequence can include a random 3-mer (e.g., GGG) that helps ensure that the poly(T) capture domain hybridizes to an mRNA. In some embodiments, an anchoring sequence can be VN, N, or NN. Alternatively, the sequence can be designed using a specific sequence of nucleotides. In some embodiments, the anchor sequence is at the 3′ end of the capture domain. In some embodiments, the anchor sequence is at the 5′ end of the capture domain.

In some embodiments, capture domains of capture probes are blocked prior to contacting the biological sample with the array, and blocking probes are used when the nucleic acid in the biological sample is modified prior to its capture on the array. In some embodiments, the blocking probe is used to block or modify the free 3′ end of the capture domain. In some embodiments, blocking probes can be hybridized to the capture probes to mask the free 3′ end of the capture domain, e.g., hairpin probes, partially double stranded probes, or complementary sequences. In some embodiments, the free 3′ end of the capture domain can be blocked by chemical modification, e.g., addition of an azidomethyl group as a chemically reversible capping moiety such that the capture probes do not include a free 3′ end. Blocking or modifying the capture probes, particularly at the free 3′ end of the capture domain, prior to contacting the biological sample with the array, prevents modification of the capture probes, e.g., prevents the addition of a poly(A) tail to the free 3′ end of the capture probes.

Non-limiting examples of 3′ modifications include dideoxy C-3′ (3′-ddC), 3′ inverted dT, 3′ C3 spacer, 3 Amino, and 3′ phosphorylation. In some embodiments, the nucleic acid in the biological sample can be modified such that it can be captured by the capture domain. For example, an adaptor sequence (including a binding domain capable of binding to the capture domain of the capture probe) can be added to the end of the nucleic acid, e.g., fragmented genomic DNA. In some embodiments, this is achieved by ligation of the adaptor sequence or extension of the nucleic acid. In some embodiments, an enzyme is used to incorporate additional nucleotides at the end of the nucleic acid sequence, e.g., a poly(A) tail. In some embodiments, the capture probes can be reversibly masked or modified such that the capture domain of the capture probe does not include a free 3′ end. In some embodiments, the 3′ end is removed, modified, or made inaccessible so that the capture domain is not susceptible to the process used to modify the nucleic acid of the biological sample, e.g., ligation or extension.

In some embodiments, the capture domain of the capture probe is modified to allow the removal of any modifications of the capture probe that occur during modification of the nucleic acid molecules of the biological sample. In some embodiments, the capture probes can include an additional sequence downstream of the capture domain, e.g., 3′ to the capture domain, namely a blocking domain.

In some embodiments, the capture domain of the capture probe can be a non-nucleic acid domain. Examples of suitable capture domains that are not exclusively nucleic-acid based include, but are not limited to, proteins, peptides, aptamers, antigens, antibodies, and molecular analogs that mimic the functionality of any of the capture domains described herein.

Unique Molecular Identifier

The capture probe can include one or more (e.g., two or more, three or more, four or more, five or more) Unique Molecular Identifiers (UMIs). A unique molecular identifier is a contiguous nucleic acid segment or two or more non-contiguous nucleic acid segments that function as a label or identifier for a particular analyte, or for a capture probe that binds a particular analyte (e.g., via the capture domain).

A UMI can be unique. A UMI can include one or more specific polynucleotides sequences, one or more random nucleic acid andor amino acid sequences, andor one or more synthetic nucleic acid andor amino acid sequences.

In some embodiments, the UMI is a nucleic acid sequence that does not substantially hybridize to analyte nucleic acid molecules in a biological sample. In some embodiments, the UMI has less than 80% sequence identity (e.g., less than 70%, 60%, 50%, or less than 40% sequence identity) to the nucleic acid sequences across a substantial part (e.g., 80% or more) of the nucleic acid molecules in the biological sample. These nucleotides can be completely contiguous, i.e., in a single stretch of adjacent nucleotides, or they can be separated into two or more separate subsequences that are separated by 1 or more nucleotides.

In some embodiments, a UMI is attached to an analyte in a reversible or irreversible manner. In some embodiments, a UMI is added to, for example, a fragment of a DNA or RNA sample before, during, andor after sequencing of the analyte. In some embodiments, a UMI allows for identification andor quantification of individual sequencing-reads. In some embodiments, a UMI is a used as a fluorescent barcode for which fluorescently labeled oligonucleotide probes hybridize to the UMI.

### Methods of the Disclosure

As outlined above, some aspects of the disclosure provide methods for preparing a high-density array of target features, as well as methods for manufacturing devices useful for spatial array-based analysis of biological samples. Also provided in some embodiments of the disclosure are methods for spatial array-based analysis of biological samples.

Methods of Preparing Dense Arrays of Target Features

Methods of preparing a high-density array of target features are described. In some embodiments, the method includes providing a substrate on or adjacent to a permanent magnet that can generate a first magnetic field. The substrate can include a first surface and a second surface (e.g., parallel to the first surface). As described above, the first surface can include a plurality of trapping regions (e.g., array of trapping regions) that can include one or more of magnetic micro-features, micro-wells and chemical coating. In some embodiments, the plurality of the magnetic micro-features may include a chemical coating on the surface of the magnetic micro-features. In some embodiments, the plurality of the micro-wells may include a chemical coating on the surface of the micro-wells. The method can further include receiving, by the first surface of the substrate, a sample (e.g., a solution) that can include a plurality of target features. For example, the substrate can be coupled (e.g., releasably attached) to a microfluidic system that can transport the sample and introduce the sample on the first surface of the substrate.

In some embodiments, the trapping region can include a high density array of magnetic micro-features. In some embodiments, the method can include fabricating the magnetic micro-features on the first surface (e.g., by electroplating, template electrodeposition, etc.). The method can include generating, by the various magnetic micro-features in the trapping region, a second magnetic field based on the interaction of the magnetic micro-features with the first magnetic field of the permanent magnet. For example, the magnetic micro-features can include ferromagnetic material that can get magnetized in the presence of an external magnetic field (e.g., first magnetic field of the permanent magnet). The magnetization can involve alignment of magnetic dipoles in the magnetic micro-features that can result in the generation of the second magnetic field. The second magnetic field can have a higher gradient than the first magnetic field, and may apply a stronger magnetic force on a magnetic feature (e.g., a magnetic target feature) relative to the magnetic force applied by the first magnetic field. The second magnetic field can be localized around the micro-features. In other words, the strength of the second magnetic field can have large spatial variation (e.g., relative to the spatial variation of the strength of first magnetic field) near the magnetic micro-features.

In some embodiments, the method can further include driving a target feature (of the plurality of target features) by one or more of a first magnetic force (due to the interaction between the first magnetic field and the target feature) and the second magnetic force (due to interaction between the second magnetic field and the target particle) towards the magnetic micro-feature. Based on the distance of the target feature from the magnetic micro-features, the first magnetic force or the second magnetic force can be the dominant force in driving the target particle. For example, the second magnetic field can be much weaker than the first magnetic field at large distances from the magnetic micro-feature. As a result, at these large distances, the target feature is primarily driven by the first magnetic force. At short distance from the magnetic micro-feature, the target feature can be primarily driven by the second magnetic force towards the magnetic micro-feature and can capture the target feature at the magnetic micro-feature. At short distances, the gradient of the second magnetic field can be much larger than the gradient of the first magnetic field resulting in stronger second magnetic force. The first andor the second magnetic force can drive other target features (of the plurality of target features) to the array of magnetic micro-features. The various target features can be captured by the array of micro-features, thereby forming a spatial array of target features.

The trapping region can include a high density array of micro-wells etched on the first surface of the substrate. In some embodiments, the method can include driving one or more target features towards a micro-well by the first magnetic force. This can result in the formation of an array of target features captured in the array of micro-wells in the first surface.

In some embodiments, the micro-wells can include magnetic micro-features. The method can include driving a target feature towards the magnetic micro-wells by the first magnetic force. The target feature can be captured in the micro-well by the first magnetic force andor the second magnetic force. The trapping regions can include an array of micro-wells including magnetic micro-features. In some embodiments, the method can further include immobilizing the target feature in the micro-well by a chemical coating, overlaid on the micro-feature andor the wall of the micro-well. In some embodiments, the chemical coating of the magnetic micro-feature can include an attachment moiety capable of binding to and immobilizing the target feature.

As described above, the target feature can include a capture probe comprising a spatial barcode and a capture domain. In some embodiments, the method can include binding the capture probe to an analyte (e.g., in a biological sample). In some embodiments, the method can further include associating the target feature with its location on the substrate. For example, the associating can include determining the sequence (sequencing) of the spatial barcode of the target feature and associating the determined spatial barcode sequence with the location of the target feature on the substrate. The sequencing can include in-situ sequencing such as sequencing-by-synthesis (SBS), sequential fluorescence hybridization, sequencing by ligation, nucleic acid hybridization, or high-throughput digital sequencing techniques.

After the array of target particles has been prepared (e.g., based on the method described above), the array can be used for spatial analysis of a biological sample. In some embodiments, spatial analysis of a biological sample can be performed by providing the spatial array of target features (“spatial array”) and contacting the biological sample to the spatial array. The contacting can be performed under conditions that can allow for the biological analyte in the biological sample to bind with the capture probe on the target feature. The location of the analyte on the surface of the substrate can be determined based on the binding of the analyte to the capture probe. The determined location can be used to identify the location of the analyte in the biological sample.

In some embodiments, spatial analysis can include determining all or a part of the sequence of the biological analyte specifically bound to the capture domain, or a complement thereof. In some embodiments, spatial analysis can include the sequence of the spatial barcode, or a complement thereof, and using the determined sequence to identify the location of the analyte in the biological sample. Various methods of spatial analysis of analytes of a biological sample using an array of target features on a substrate are described below.

Methods for Spatial Analysis of Biological Analytes

As described in greater detail below, the devices disclosed herein can be used for spatial array-based analysis of biological samples. An exemplary workflow for the array-based spatial analysis methods involves the transfer of one or more analytes from a biological sample to an array of features on a substrate, where each feature is associated with a unique spatial location on the array. Subsequent analysis of the transferred analytes includes determining the identity of the analytes and the spatial location of each analyte within the biological sample. The spatial location of each analyte within the biological sample is determined based on the feature to which each analyte is bound on the array, and the feature's relative spatial location within the array.

In some embodiments, provided herein are methods for spatial analysis of a biological analyte in a biological sample, the methods include: (a) providing a device of the disclosure; (b) contacting the device with the biological sample under conditions wherein the biological analyte binds to a capture probe coupled to a target feature of the device; and (c) determining (i) all or a part of the sequence of the biological analyte bound to the first capture probe, or a complement thereof, and (ii) the sequence of the spatial barcode, or a complement thereof, and using the determined sequence of (i) and (ii) to identify the location of the biological analyte in the biological sample.

Biological Samples

As outlined above, samples containing an analyte for spatial analysis include biological samples. In some embodiments, a biological sample is obtained from a subject for analysis using any of a variety of techniques including, but not limited to, biopsy, surgery, and laser capture microscopy (LCM), and generally includes cells or nuclei andor other biological material from the subject. In addition to the subjects described above, a biological sample can be obtained from non-mammalian organisms (e.g., a plants, an insect, an arachnid, a nematode (e.g., Caenorhabditis elegans), a fungi, an amphibian, or a fish (e.g., zebrafish)). In some embodiments, a biological sample can be obtained from a prokaryote such as a bacterium, e.g., Escherichia coli, Staphylococci or Mycoplasma pneumoniae; an archaea; a virus such as Hepatitis C virus or human immunodeficiency virus; or a viroid. In some embodiments, a biological sample can be obtained from a eukaryote, such as a patient derived organoid (PDO) or patient derived xenograft (PDX). In some embodiments, a biological sample can include organoids, a miniaturized and simplified version of an organ produced in vitro in three dimensions that shows realistic micro-anatomy. Organoids can be generated from one or more cells from a tissue, embryonic stem cells, andor induced pluripotent stem cells, which can self-organize in three-dimensional culture owing to their self-renewal and differentiation capacities. In some embodiments, an organoid is a cerebral organoid, an intestinal organoid, a stomach organoid, a lingual organoid, a thyroid organoid, a thymic organoid, a testicular organoid, a hepatic organoid, a pancreatic organoid, an epithelial organoid, a lung organoid, a kidney organoid, a gastruloid, a cardiac organoid, or a retinal organoid. Subjects from which biological samples can be obtained can be healthy or asymptomatic individuals, individuals that have or are suspected of having a disease (e.g., cancer) or a pre-disposition to a disease, andor individuals that are in need of therapy or suspected of needing therapy.

In some embodiments, a biological sample can include a single analyte of interest, or more than one analyte of interest. Methods for performing multiplexed assays to analyze two or more different analytes in a single biological sample is discussed in a subsequent section of this disclosure.

A variety of steps can be performed to prepare a biological sample for analysis. Except where indicated otherwise, preparative steps can generally be combined in any manner to appropriately prepare a particular sample for analysis.

In some embodiments, the biological sample can be preserved after completion of an assay with a feature or arrangement of features for additional rounds of spatial detection of analytes. In some embodiments, the biological sample, features, array, or any combination thereof can be preserved after the spatial profiling. In some embodiments, the biological sample, features, array, or combinations thereof can be protected from dehydration (e.g., drying, desiccation). In some embodiments, the biological sample, features, array, or combinations thereof, can be protected from evaporation. Methods of preserving andor protecting biological samples, features, or arrays are known in the art. For example, in a non-limiting way, the biological sample, features, array, or combinations thereof can be covered by a reversible sealing agent. Any suitable reversible sealing agent can be used. Methods of reversible sealing are known in the art (See, e.g., WO 2019104337, which is incorporated herein by reference). In a non-limiting way, suitable reversible sealing agents can include non-porous materials, membranes, lids, or oils (e.g., silicone oil, mineral oil). In further non-limiting examples, the biological sample, features, array, or combinations thereof can be preserved in an environmental chamber (e.g., hermetically sealed) and removed for additional rounds of spatial analysis at a later time.

Biological Analytes

The devices and methods described in this disclosure can be used to detect and analyze a wide variety of different analytes. For the purpose of this disclosure, an analyte of the disclosure will be understood to include any biological substance, structure, moiety, or component to be analyzed.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or 0-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte can be an organelle (e.g., nuclei or mitochondria).

Cell surface features corresponding to analytes can include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Analytes can be derived from a specific type of biological particles, including a cell andor a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis.

Examples of nucleic acid analytes include DNA analytes such as genomic DNA, methylated DNA, specific methylated DNA sequences, fragmented DNA, mitochondrial DNA, in situ synthesized PCR products, and RNADNA hybrids.

Examples of nucleic acid analytes also include RNA analytes such as various types of coding and non-coding RNA. Examples of the different types of RNA analytes include messenger RNA (mRNA), ribosomal RNA (rRNA), transfer RNA (tRNA), microRNA (miRNA), and viral RNA. The RNA can be a transcript (e.g., present in a tissue section). The RNA can be small (e.g., less than 200 nucleic acid bases in length) or large (e.g., RNA greater than 200 nucleic acid bases in length). The RNA can be double-stranded RNA or single-stranded RNA. The RNA can be circular RNA. The RNA can be a bacterial rRNA (e.g., 16S rRNA or 23S rRNA).

Additional examples of analytes include mRNA and cell surface features (e.g., using the labelling agents described herein), mRNA and intracellular proteins (e.g., transcription factors), mRNA and cell methylation status, mRNA and accessible chromatin (e.g., ATAC-seq, DNase-seq, andor MNase-seq), mRNA and metabolites (e.g., using the labelling agents described herein), a barcoded labelling agent (e.g., the oligonucleotide tagged antibodies described herein) and a V(D)J sequence of an immune cell receptor (e.g., T-cell receptor), mRNA and a perturbation agent (e.g., a CRISPR crRNAsgRNA, TALEN, zinc finger nuclease, andor antisense oligonucleotide as described herein). In some embodiments, a perturbation agent can be a small molecule, an antibody, a drug, an aptamer, a miRNA, a physical environmental (e.g., temperature change), or any other known perturbation agents.

Analytes can include a nucleic acid molecule with a nucleic acid sequence encoding at least a portion of a V(D)J sequence of an immune cell receptor (e.g., a TCR or BCR). In some embodiments, the nucleic acid molecule is cDNA first generated from reverse transcription of the corresponding mRNA, using a poly(T) containing primer. The generated cDNA can then be barcoded using a capture probe, featuring a barcode sequence (and optionally, a UMI sequence) that hybridizes with at least a portion of the generated cDNA. In some embodiments, a template switching oligonucleotide hybridizes to a poly(C) tail added to a 3-end of the cDNA by a reverse transcriptase enzyme. The original mRNA template and template switching oligonucleotide can then be denatured from the cDNA and the barcoded capture probe can then hybridize with the cDNA and a complement of the cDNA generated. Additional methods and compositions suitable for barcoding cDNA generated from mRNA transcripts including those encoding V(D)J regions of an immune cell receptor andor barcoding methods and composition including a template switch oligonucleotide are described in PCT Patent Application PCTUS2017057269. V(D)J analysis can also be completed with the use of one or more labelling agents that bind to particular surface features of immune cells and associated with barcode sequences. The one or more labelling agents can include an MEW or MEW multimer.

As described above, the analyte can include a nucleic acid capable of functioning as a component of a gene editing reaction, such as, for example, clustered regularly interspaced short palindromic repeats (CRISPR)-based gene editing. Accordingly, the capture probe can include a nucleic acid sequence that is complementary to the analyte (e.g., a sequence that can hybridize to the CRISPR RNA (crRNA), single guide RNA (sgRNA), or an adapter sequence engineered into a crRNA or sgRNA).

In certain embodiments, an analyte can be extracted from a live cell. Processing conditions can be adjusted to ensure that a biological sample remains live during analysis, and analytes are extracted from (or released from) live cells of the sample. Live cell-derived analytes can be obtained only once from the sample, or can be obtained at intervals from a sample that continues to remain in viable condition.

In general, the devices and methods disclosed herein can be used to analyze any number of analytes. Methods for performing multiplexed assays to analyze two or more different analytes will be discussed in a subsequent section of this disclosure.

As outlined above, the methods for spatial analysis of biological analytes disclosed herein generally include a step of identifying the location of a biological analyte in a biological sample. There are at least two general methods to associate a spatial barcode with one or more neighboring biological particles (e.g., cells, cell beads, or nuclei), such that the spatial barcode identifies the one or more biological particles, andor contents of the one or more biological particles, as associated with a particular spatial location. One general method is to provide conditions sufficient to facilitate the movement (e.g., diffusion) of analytes from or out of a biological particle (e.g., cell, cell bead, or nucleus) and towards a spatially-barcoded array. For example, a spatially-barcoded array populated with capture probes (as described further herein) can be contacted with a biological sample, followed by permeabilization of the biological sample to allow the analyte to migrate away from the sample and toward the array where it interacts with the capture probes. The analyte-bound capture probes can then be analyzed in order to obtain spatially-resolved information about the sample from which the analyte originated.

An exemplary workflow can include preparing a biological sample on a spatially-barcoded array. Sample preparation can include contacting the sample (e.g., a plurality of biological particles, such as cells, cell beads, or nuclei) with a slide, fixing the sample, andor staining the biological sample for imaging. The stained sample can be then imaged on the array using brightfield (to image the sample hematoxylin and eosin stain) andor fluorescence (to image features) modalities in such a way that positions in the spatially-barcoded array can be mapped to different biological particles in the sample (or samples) contacted with the slide. Optionally, the sample can be destained prior to permeabilization. In some embodiments, analytes are then released from the sample and capture probes forming the spatially-barcoded array hybridize or bind the released analytes. The analyte-bound capture probes can then be analyzed to determine the identity of the analyte and where it was located on the spatial array. Where the analyte is RNA (e.g., mRNA), the RNA can be reverse transcribed into cDNA containing information from the spatial barcode of the capture probe bound to the RNA, and an amplicon library can be prepared and sequenced to identify the RNA and where it was located on the spatial array. The mapping of positions in the spatially-barcoded array to different biological particles in the sample (or samples) contacted with the slide can then be used to provide information about the analytes from the different biological particles from the sample (or samples).

The capture probes can be optionally cleaved from the array, and the captured analytes can be spatially-barcoded by performing a reverse transcriptase first strand cDNA reaction. A first strand cDNA reaction can be optionally performed using template switching oligonucleotides. For example, a template switching oligonucleotide can hybridize to a poly(C) tail added to a 3′ end of the cDNA by a reverse transcriptase enzyme in a template independent manner. The original mRNA template and template switching oligonucleotide can then be denatured from the cDNA and the spatially-barcoded capture probe can then hybridize with the cDNA and a complement of the cDNA can be generated. The first strand cDNA can then be purified and collected for downstream amplification steps. The first strand cDNA can be amplified using PCR, where the forward and reverse primers flank the spatial barcode and analyte regions of interest, generating a library associated with a particular spatial barcode. In some embodiments, the cDNA comprises a sequencing by synthesis (SBS) primer sequence. The library amplicons are sequenced and analyzed to decode spatial information.

Another general method is to cleave spatially-barcoded capture probes from an array, and promote the spatially-barcoded capture probes towards andor into or onto a biological particle from a biological sample that has been contacted with the array. For example, a spatially-barcoded array populated with capture probes (as described further herein) can be contacted with a sample, followed by cleavage of the spatially-barcoded capture probes to allow them to interact with biological particles (e.g., cells, cell beads, or nuclei) from the provided biological sample. The interaction can be a covalent or non-covalent cell-surface interaction. The interaction can be an intracellular interaction facilitated by a delivery system or a cell penetration peptide. Once the spatially-barcoded capture probe is associated with a particular biological particle (e.g., cell, cell bead, or nucleus), the sample can be optionally removed for analysis. Once the tagged cell is associated with the spatially-barcoded capture probe, the capture probes can be analyzed to obtain spatially-resolved information about the tagged cell.

Using the methods, compositions, kits, and devices described herein, RNA transcripts present in biological samples (e.g., biological particles such as cells, cell beads, or nuclei) can be analyzed using spatial transcriptome analysis. In particular, in some cases, the barcoded oligonucleotides can be configured to prime, replicate, and consequently yield barcoded extension products from an RNA template, or derivatives thereof. For example, in some cases, the barcoded oligonucleotides may include mRNA specific priming sequences, e.g., poly-T primer segments that allow priming and replication of mRNA in a reverse transcription reaction or other targeted priming sequences. Alternatively or additionally, random RNA priming can be carried out using random N-mer primer segments of the barcoded oligonucleotides. Reverse transcriptases (RTs) can use an RNA template and a primer complementary to the 3′ end of the RNA template to direct the synthesis of the first strand complementary DNA (cDNA). Many RTs can be used in this reverse transcription reactions, including, for example, avian myeloblastosis virus (AMV) reverse transcriptase, moloney murine leukemia virus (M-MuLV or MMLV), and other variants thereof. Some recombinant M-MuLV reverse transcriptase, such as, for example, PROTOSCRIPT® II reverse transcriptase, can have reduced RNase H activity and increased thermostability when compared to its wild type counterpart, and provide higher specificity, higher yield of cDNA and more full-length cDNA products with up to 12 kilobase (kb) in length. In some embodiments, the reverse transcriptase enzyme is a mutant reverse transcriptase enzyme such as, but not limited to, mutant MMLV reverse transcriptase. In another embodiment, the reverse transcriptase is a mutant MMLV reverse transcriptase such as, but not limited to, one or more variants described in US Patent Publication No. 20180312822 and U.S. Provisional Patent Application No. 62/946,885 filed on Dec. 11, 2019.

In a non-limiting example of the workflows described above, a biological sample (e.g., a plurality of biological particles, such as cells or nuclei), can be fixed with methanol, stained with hematoxylin and eosin, and imaged. Optionally, the sample can be destained prior to permeabilization. The images can be used to map gene expression patterns to biological particles from the biological sample. A permeabilization enzyme can be used to permeabilize the biological sample directly on the slide. Analytes (e.g., polyadenylated mRNA) released from the biological particles (e.g., cells or nuclei) of the biological sample can be captured by capture probes within a capture area on a substrate. Reverse transcription (RT) reagents can be added to permeabilized biological samples. Incubation with the RT reagents can produce spatially-barcoded full-length cDNA from the captured analytes (e.g., polyadenylated mRNA). Second strand reagents (e.g., second strand primers, enzymes) can be added to the biological sample on the slide to initiate second strand synthesis. The resulting cDNA can be denatured from the capture probe template and transferred (e.g., to a clean tube) for amplification, andor library construction. The spatially-barcoded, full-length cDNA can be amplified via PCR prior to library construction. The cDNA can then be enzymatically fragmented and size-selected in order to optimize the cDNA amplicon size. P5, P7, i7, and i5 can be used as sample indexes, and TruSeq Read 2 can be added via End Repair, A-tailing, Adaptor Ligation, and PCR. The cDNA fragments can then be sequenced using paired-end sequencing using TruSeq Read 1 and TruSeq Read 2 as sequencing primer sites.

In some embodiments, performing correlative analysis of data produced by this workflow, and other workflows described herein, can yield over 95% correlation of genes expressed across two capture areas (e.g., 95% or greater, 96% or greater, 97% or greater, 98% or greater, or 99% or greater). When performing the described workflows using single cell RNA sequencing of nuclei, in some embodiments, correlative analysis of the data can yield over 90% (e.g., over 90%, 91%, 92%, 93%, 94%, 95%, 96%, 97%, 98%, or 99%) correlation of genes expressed across two capture areas.

In some embodiments, after cDNA is generated (e.g., by reverse transcription) the cDNA can be amplified directly on the substrate surface. Generating multiple copies of the cDNA (e.g., cDNA synthesized from captured analytes) via amplification directly on the substrate surface can improve final sequencing library complexity. Thus, in some embodiments, cDNA can be amplified directly on the substrate surface by isothermal nucleic acid amplification. In some embodiments, isothermal nucleic acid amplification can amplify RNA or DNA.

In some embodiments, provided herein are methods for spatially detecting an analyte (e.g., detecting the location of an analyte, e.g., a biological analyte) from a biological sample (e.g., an analyte present in a biological sample, such as one or more biological particles, e.g., cells, cell beads, or nuclei) that include: (a) providing a biological sample on a substrate; (b) staining the biological sample on the substrate, imaging the stained biological sample, and selecting the biological sample or subsection of the biological sample (e.g., region of interest) to subject to analysis; (c) providing an array comprising one or more pluralities of capture probes on a substrate; (d) contacting the biological sample with the array, thereby allowing a capture probe of the one or more pluralities of capture probes to capture the analyte of interest; and (e) analyzing the captured analyte, thereby spatially detecting the analyte of interest.

Any variety of staining and imaging techniques as described herein or known in the art can be used in accordance with methods described herein. In some embodiments, the staining includes optical labels as described herein, including, but not limited to, fluorescent, radioactive, chemiluminescent, calorimetric, or colorimetric detectable labels. In some embodiments, the staining includes a fluorescent antibody directed to a target analyte (e.g., cell surface or intracellular proteins) in the biological sample. In some embodiments, the staining includes an immunohistochemistry stain directed to a target analyte (e.g., cell surface or intracellular proteins) in the biological sample. In some embodiments, the staining includes a chemical stain such as hematoxylin and eosin (H&E) or periodic acid-schiff (PAS). In some embodiments, significant time (e.g., days, months, or years) can elapse between staining andor imaging the biological sample and performing analysis. In some embodiments, reagents for performing analysis are added to the biological sample before, contemporaneously with, or after the array is contacted to the biological sample. In some embodiments, step (d) includes depositing biological particles (e.g., cells, cell beads, or nuclei) from the biological sample onto the array. In some embodiments, the array is a flexible array where the plurality of spatially-barcoded features (e.g., a substrate with capture probes, a bead with capture probes) are attached to a flexible substrate. In some embodiments, measures are taken to slow down a reaction (e.g., cooling the temperature of the biological sample or using enzymes that preferentially perform their primary function at lower or higher temperature as compared to their optimal functional temperature) before the biological sample is contacted with the array. In some embodiments, step (e) is performed without bringing the biological sample out of contact with the array. In some embodiments, step (e) is performed after the biological sample is no longer in contact with the array. In some embodiments, the biological sample is tagged with an analyte capture agent before, contemporaneously with, or after staining andor imaging of the biological sample. In such cases, significant time (e.g., days, months, or years) can elapse between staining andor imaging and performing analysis. In some embodiments, the array is adapted to facilitate biological analyte migration from the stained andor imaged biological sample onto the array (e.g., using any of the materials or methods described herein). In some embodiments, a biological sample is permeabilized before being contacted with an array. In some embodiments, the rate of permeabilization is slowed prior to contacting a biological sample with an array (e.g., to limit diffusion of analytes away from their original locations in the biological sample). In some embodiments, modulating the rate of permeabilization (e.g., modulating the activity of a permeabilization reagent) can occur by modulating a condition that the biological sample is exposed to (e.g., modulating temperature, pH, andor light). In some embodiments, modulating the rate of permeabilization includes use of external stimuli (e.g., small molecules, enzymes, andor activating reagents) to modulate the rate of permeabilization. For example, a permeabilization reagent can be provided to a biological sample prior to contact with an array, which permeabilization reagent is inactive until a condition (e.g., temperature, pH, andor light) is changed or an external stimulus (e.g., a small molecule, an enzyme, andor an activating reagent) is provided.

In some embodiments, provided herein are methods for spatially detecting an analyte (e.g., detecting the location of an analyte, e.g., a biological analyte) from a biological sample (e.g., present in biological particles, such as cell, cell bead, or nuclei, from a biological sample) that include: (a) providing a biological sample on a substrate; (b) staining the biological sample on the substrate, imaging the stained biological sample, and selecting the biological sample or subsection of the biological sample (e.g., a region of interest) to subject to spatial transcriptomic analysis; (c) providing an array comprising one or more pluralities of capture probes on a substrate; (d) contacting the biological sample with the array, thereby allowing a capture probe of the one or more pluralities of capture probes to capture the biological analyte of interest; and (e) analyzing the captured biological analyte, thereby spatially detecting the biological analyte of interest.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

Additional embodiments are disclosed in further detail in the following examples, which are provided by way of illustration and are not in any way intended to limit the scope of this disclosure or the claims.

### EXAMPLES

Additional embodiments are disclosed in further detail in the following examples, which are provided by way of illustration and are not in any way intended to limit the scope of this disclosure or the claims.

### Example 1

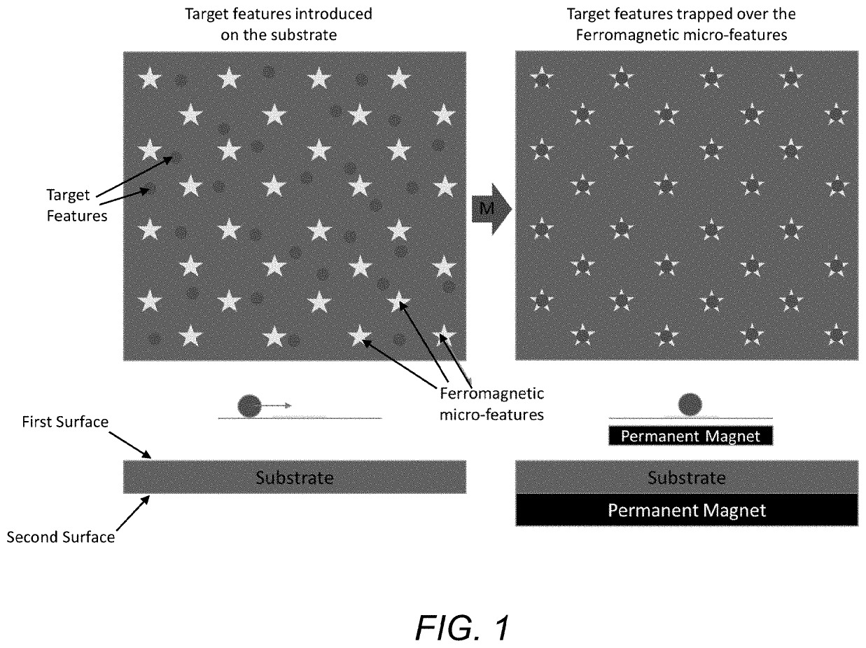

This Example describes a non-limiting exemplary method for generating an array of target features in accordance with some non-limiting embodiments of the disclosure. As illustrated in FIG. 1, the method includes a substrate that includes multiple ferromagnetic micro-features arranged in an array over a first surface of the substrate. The target features are introduced to the first surface (e.g., via a microfluidic system). In some instances, the method further includes a permanent magnet that is placed in proximity to (e.g., adjacent to) the second surface of the substrate. The permanent magnet generates a first magnetic field that magnetizes the ferromagnetic micro-features. Upon magnetization, each of the ferromagnetic micro-features generate a magnetic field that has a higher gradient than the first magnetic field and is localized around the ferromagnetic micro-features. Due to a high gradient, the magnetic fields from the ferromagnetic micro-features exerts a strong magnetic force on the target features. As a result, one or more target features can be driven towards and trapped by the ferromagnetic micro-features. If a target feature is sufficiently large (e.g., has a diameter larger than the distance between ferromagnetic micro-features), it can be captured by multiple ferromagnetic micro-features.

In this example, the ferromagnetic micro-features are fabricated on the first surface by using existing fabrication technologies such as template electrodeposition, electroplating, etc. In some instances, the ferromagnetic micro-features can be densely packed on the first surface that can result in a dense array of trapped target features.

### Example 2

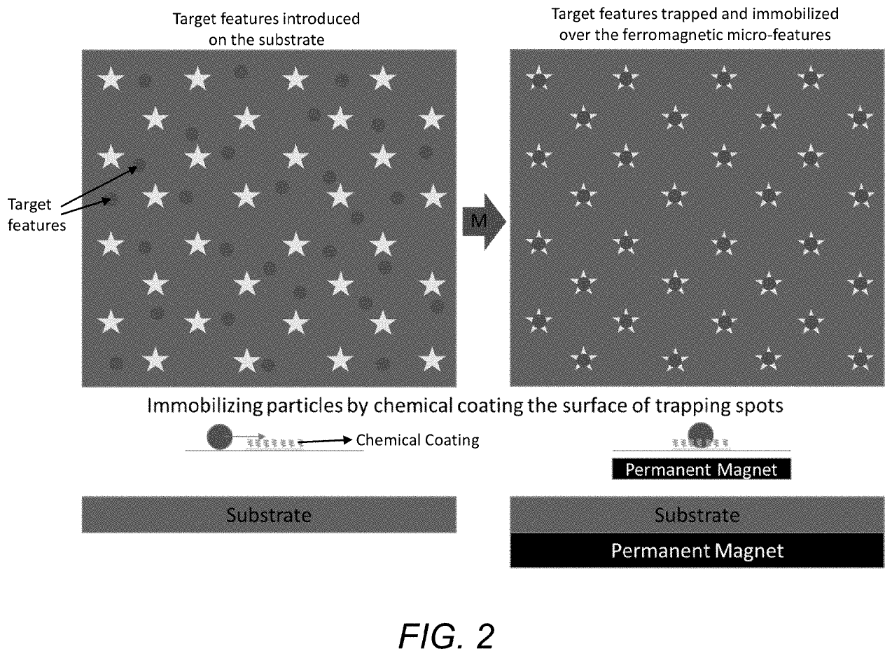

This Example describes a non-limiting exemplary implementation of the method described in Example 1 and FIG. 1 that further includes chemical coating on the surface of the ferromagnetic micro-features. In this implementation, as illustrated in FIG. 2, the chemical coating immobilizes the target features positioned over the ferromagnetic micro-features. A non-limiting example of a chemical coating includes a poly-lysine-based polymer.

### Example 3

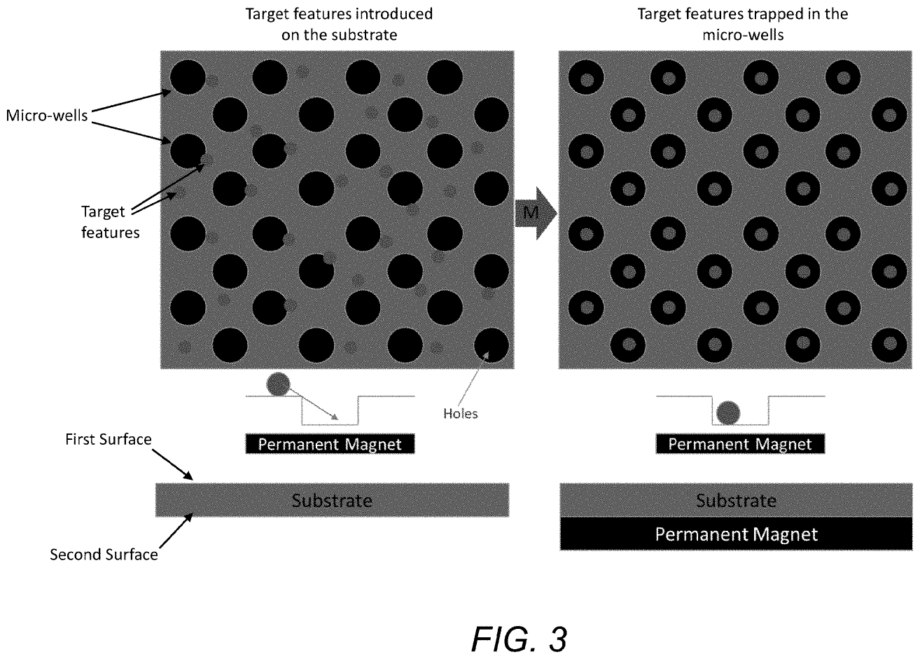

This Example describes another exemplary method for generating an array of target features in accordance with some non-limiting embodiments of the disclosure. As illustrated in FIG. 3, the method includes a substrate that includes multiple micro-wells arranged in an array over a first surface of the substrate. The target features are introduced to the first surface (e.g., via a microfluidic system). In some particular implementations, the method further includes a permanent magnet that is placed in proximity to (e.g., adjacent to) the second surface of the substrate. The permanent magnet generates a first magnetic field that can apply a force on the target features (proportional to the gradient of the first magnetic field) that can drive one or more target features into a micro-well. The one or more target features can be trapped in the micro-well.

The micro-wells are etched on the first surface by using existing micro-fabrication etching technologies. The micro-wells can be densely packed on the first surface that can result in a dense array of trapped target features.

### Example 4

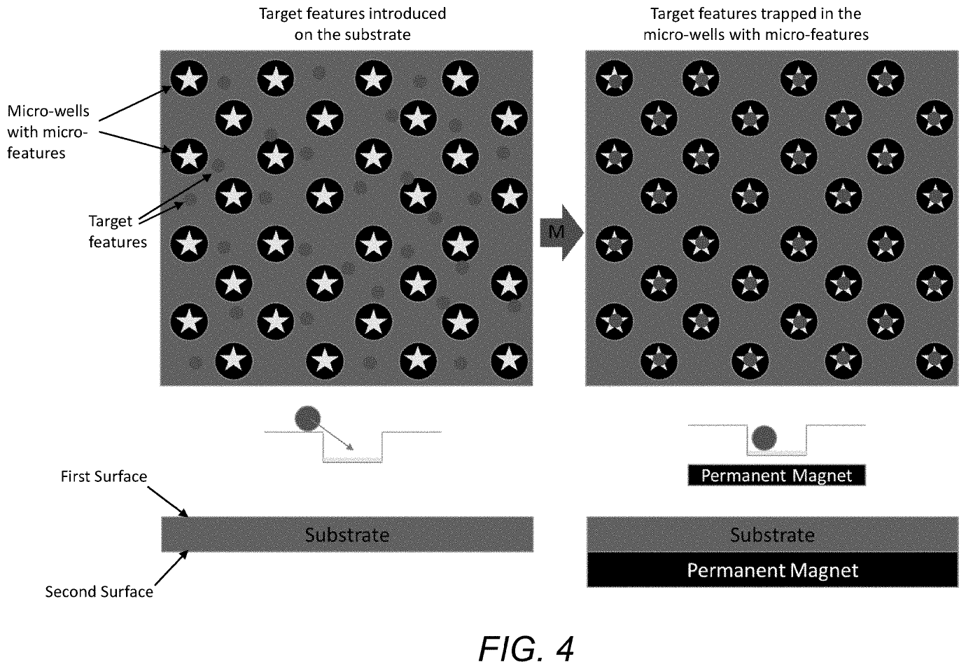

This Example describes a non-limiting exemplary implementation of the method described above in Example 3 and of FIG. 3 that further includes a ferromagnetic micro-feature in each of the micro-wells. In this implementation, as illustrated in FIG. 4, the ferromagnetic micro-feature interacts with the first magnetic field generated by the permanent magnet and generates a second magnetic field. The second magnetic field has a higher gradient than the first magnetic field and is localized in and around the micro-wells. The second magnetic field can drive and trap the one or more target features in a micro-well.

### Example 5

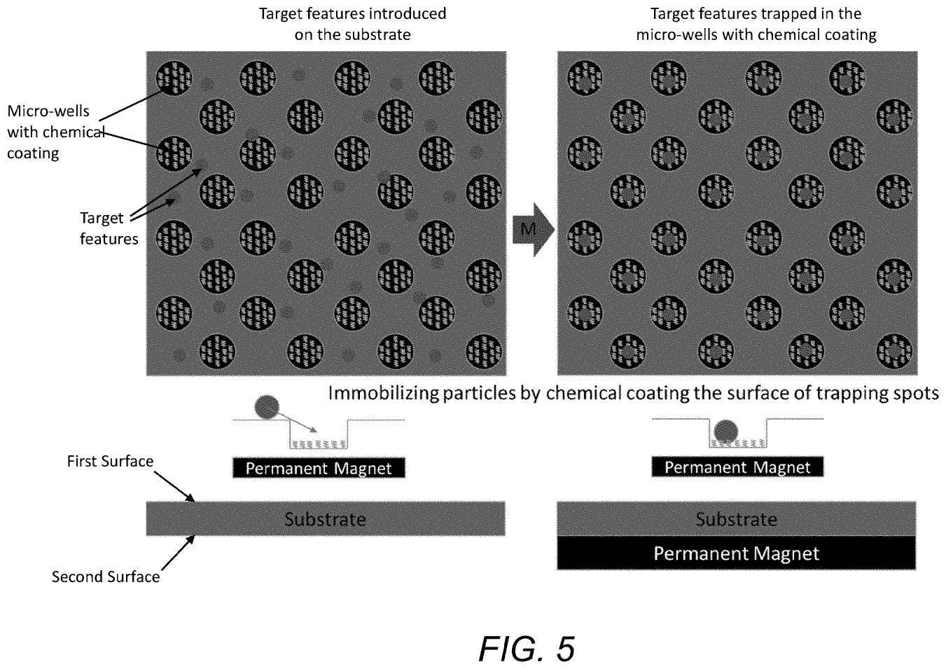

This Example describes another non-limiting exemplary implementation of the method described in Example 3 and FIG. 3 that further includes chemical coating on a surface of the micro-wells. In this implementation, as illustrated in FIG. 5, the chemical coating can be applied to, for example, a portion of the wall of a micro-well. Additionally or alternately, the chemical coating can be applied to the base of the micro-well. The chemical coating immobilizes the target feature in the micro-wells. A non-limiting example of a chemical coating is a poly-lysine based polymer.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

1. A method for preparing an array of target features comprising:
(a) providing a substrate on or adjacent to a permanent magnet configured to generate a first magnetic field, the substrate comprising a first surface and a second surface, wherein the first surface comprises a plurality of magnetic micro-features arranged along the first surface, and wherein the second surface of the substrate is adjacent to the permanent magnet;
(b) receiving, at the first surface of the substrate, a sample comprising a plurality of target features;
(c) generating, by a magnetic micro-feature of the plurality of magnetic micro-features, a second magnetic field based on the interaction of the magnetic micro-feature with the first magnetic field; and
(d) driving, by one or more of the first magnetic field and the second magnetic field, a target feature of the plurality of target features towards the magnetic micro-feature such that the target feature is associated with the magnetic micro-feature, thereby forming an array of the plurality of target features.

2. The method of claim 1, wherein the magnetic micro-feature comprises an attachment moiety capable of binding to the target feature, and wherein association of the target feature with the magnetic micro-feature comprises binding of the target feature to the attachment moiety of the magnetic micro-feature.

3. The method of claim 1, wherein the first surface comprises a plurality of micro-wells, and wherein a micro-well of the plurality of micro-wells comprises the magnetic micro-feature.

4. The method of claim 1, wherein the plurality of the magnetic micro-features comprises a chemical coating on the surface of the magnetic micro-features.

5. The method of claim 3, wherein the plurality of micro-wells comprises a chemical coating on the surface of the micro-wells.

6. The method of claim 4, wherein the chemical coating comprises a poly(ethylene glycol) (PEG)-based polymer, a poly(L-lysine) (PLL)-based polymer, a poly-L-lysine-grafted-polyethylene glycol (PLL-g-PEG), or a methacrylated gelatin (GelMA) polymer.

7. The method of claim 1, wherein the magnetic micro-feature is one of a circular shape, a rectangular shape, a square shape, a triangular shape and a star shape.

8. The method of claim 1, wherein the plurality of magnetic micro-features are arranged along the first surface in a rectangular array.

9. The method of claim 1, wherein a first gradient associated with the first magnetic field is smaller than a second gradient associated with the second magnetic field at the magnetic micro-feature.

10. The method of claim 1, further comprising coupling the substrate with a microfluidic system, wherein the sample comprising the plurality of target features is received from the microfluidic system.

11. The method of claim 1, wherein the magnetic micro-feature is a ferromagnetic micro-feature.

12. The method of claim 1, wherein the magnetic micro-feature comprises one or more of Ni and NiFe.

13. The method of claim 1, wherein the target feature is a magnetic bead or a bead coupled to a magnetic moiety.

14. The method of claim 1, wherein the first surface is substantially parallel to the second surface.

15. The method of claim 1, further comprising fabricating the substrate comprising the plurality of magnetic micro-features, the fabricating comprising one of template electrodeposition and electroplating.

16. The method of claim 1, wherein the target feature comprises a capture probe, wherein the capture probe comprises a spatial barcode and a capture domain, wherein the capture domain is capable of binding to an analyte, and wherein the spatial barcode is different for each target feature of the plurality of target features.

17. The method of claim 16, wherein the target feature has a location on the substrate, and wherein the method further comprises associating the target feature with its location on the substrate, and wherein associating the target feature with its location on the substrate comprises optionally sequencing the spatial barcode of the target feature and associating the determined spatial barcode sequence with the location of the target feature on the substrate.

18-20. (canceled)

21. The method of claim 17, wherein sequencing is performed via sequencing-by-synthesis (SBS), sequential fluorescence hybridization, sequencing by ligation, nucleic acid hybridization, or high-throughput digital sequencing techniques.

22. A device comprising:
(a) a substrate comprising a first surface and a second surface, wherein the first surface comprises a plurality of magnetic micro-features arranged along the first surface, and wherein the first surface is configured to receive a sample comprising a plurality of target features; and
(b) a permanent magnet configured to couple to the second surface of the substrate and to generate a first magnetic field, wherein a magnetic micro-feature of the plurality of magnetic micro-features is configured to generate a second magnetic field based on the interaction of the magnetic micro-feature with the first magnetic field, and
(c) wherein one or more of the first magnetic field and the second magnetic field are configured to move a target feature of the plurality of target features towards the magnetic micro-feature such that the target feature is associated with the magnetic micro-feature, thereby forming an array of the plurality of target features.

23-34. (canceled)

35. A device comprising:
(a) a substrate comprising a first surface and a second surface, wherein the first surface comprises a plurality micro-wells arranged along the first surface, and wherein the first surface is configured to receive a sample comprising a plurality of target features; and
(b) a permanent magnet configured to couple to the second surface of the substrate and to generate a first magnetic field,
(c) wherein the first magnetic field is configured to drive a target feature of the plurality of target features towards a micro-well of the plurality of micro-wells such that the target feature is associated with the micro-well, thereby forming an array of the plurality of target features.

36-45. (canceled)

46. An array prepared by the method of claim 1.

47. A method for spatial analysis of a biological analyte in a biological sample comprising:
(a) providing an array prepared by the method of claim 1;
(b) contacting the biological sample to the array under conditions wherein the biological analyte binds the capture probe on the target feature; and
(c) determining a location of the analyte on the surface of the substrate based on the binding of the analyte to the capture probe, and using the location of the analyte on the surface of the substrate to identify the location of the analyte in the biological sample.

48. The method of claim 47, wherein determining the location of the analyte in c) further comprises determining (i) all or a part of the sequence of the biological analyte specifically bound to the capture domain, or a complement thereof; and (ii) the sequence of the spatial barcode, or a complement thereof, and using the determined sequence of (i) and (ii) to identify the location of the analyte in the biological sample.
